(19) United States
(12) Patent Application Publication
(10) Pub. No.: US 2014/0242640 A1
(43) Pub. Date: Aug. 28, 2014

(54) Methods of Making Nanotechnological and Macromolecular Biomimetic Structures

(71) Applicant: Alexander SUNGUROFF, Duxbury, MA (US)

(72) Inventor: Alexander SUNGUROFF, Duxbury, MA (US)

(21) Appl. No.: 14/039,717

(22) Filed: Sep. 27, 2013

Related U.S. Application Data
(63) Continuation of application No. 13/757,383, filed on Feb. 1, 2013, now abandoned.
Application No. 13/757,383 is a continuation of application No. 11/636,723, filed on Dec. 11, 2006, now abandoned.
Application No. 11/636,723 is a continuation-in-part of application No. PCT/US05/20037, filed on Jun. 9, 2005.
(60) Provisional application No. 60/578,077, filed on Jun. 9, 2004.
(60) Provisional application No. 60/638,172, filed on Dec. 23, 2004.

(51) Int. Cl. C12P 19/04; C12P 7/62

(52) U.S. Cl. CPC C12P 19/04; C12P 7/62
USPC 435/101; 435/317.1; 536/23.1; 536/56; 435/252.33; 435/135; 528/361

(57) ABSTRACT

The present invention is in the fields of nanotechnology and biomimetics. In particular, the present invention relates to the use of modified ribosomes to produce biomimetic structures. These biomimetic structures, also known as directed element polymers, are not produced by traditional industrial means but instead are produced by living systems comprising modified ribosomes.

### CROSS REFERENCE TO RELATED APPLICATIONS

This application is a continuation of U.S. application Ser. No. 13/757,383, filed Feb. 1, 2013, which is a continuation of U.S. application Ser. No. 11/636,723, filed Dec. 11, 2006, which is a continuation-in-part of PCT Appl. No. PCT/US05/20037, filed Jun. 9, 2005, which claims the benefit of U.S. Provisional Appl. No. 60/578,077, filed Jun. 9, 2004 and U.S. Provisional Appl. No. 60/638,172, filed Dec. 23, 2004, each of which is incorporated herein by reference.

### BACKGROUND OF THE INVENTION

1. Field of the Invention

The present invention is in the fields of nanotechnology and biomimetics. In particular, the present invention relates to the use of modified ribosomes to produce biomimetic structures. These biomimetic structures, also known as directed element polymers, are not produced by traditional industrial means but instead are produced by living systems comprising modified ribosomes.

2. Background Art

1. Evolutionary Conservation of Ribosome Structure and Function

In all cells, deoxyribonucleic acid (DNA) records the information required for running the cell and eventually passes this information to subsequent cell generations. Cells extract the information contained within DNA through the processes of transcription and translation. During transcription, DNA is transcribed into messenger RNA (mRNA). During translation, ribosomes translate the mRNA into amino acids and assemble the amino acids into proteins for use in cellular structures and functions.

Ribosomes are present in both prokaryotic and eukaryotic cells and depending on the cell type are free-floating in the cytoplasm, are bound to endoplasmic reticulum, and/or are located within mitochondria and chloroplasts. Ribosomes found in nature are composed of a large and a small subunit. Each subunit is composed of ribosomal nucleic acid (rRNA) and protein. While prokaryotic and eukaryotic ribosomes possess subunits of different size and composition, the two are similar in structure and function. (See, e.g. Wilson and Nierhaus, Angew, Chem. Int. Ed. 42: 3464-3486 (2003); Fromont-Racine et al., Gene 313: 17-42 (2003).

A typical translation reaction involves the formation of a complex between a ribosome, a mRNA codon (a unit of 3 nucleotides), a tRNA covalently linked to one of 20 naturally occurring amino acids (an aminoacyl-tRNA), a tRNA covalently linked to the peptide chain being elongated by translation (a peptidyl-tRNA), and associated factors. A particular mRNA codon is recognized by an aminoacyl-tRNA possessing a complementary sequence (an anti-codon). The ribosome executes its task of building of an ordered sequence of polymerized amino acids, i.e. a protein, by positioning each mRNA, aminoacyl-tRNA and peptidyl-tRNA within specific ribosomal sites such that a bond can be catalyzed between an amino acid and the growing peptide chain. (See, e.g., Rodnina and Wintermeyer, Curr. Opin. Struct. Biol. 13:334-340 (2003); Wilson and Nierhaus Angew. Chem. Int. Ed. 42: 3464-3486 (2003); Agris, Nucleic Acids Res. 32: 223-238 (2004); Youngman et al., Cell 117: 589-599 (2004).

The ribosome is so central to the survival of all living cells that over evolutionary timeframes ribosomes and associated tRNA molecules have been strongly conserved. This is because any radical change to these mechanisms is too traumatic to the cell and results in cell death. It appears that only through human-mediated engineering can the ribosome/tRNA machinery leap over the dead-zones of non-viable states to achieve functioning systems that are radically different from the highly conserved mechanisms of naturally evolved life. Creating such in vitro systems that produce biomimetic products as a result of the engineering is described herein.

2. Modified Ribosomes

Much information has been gained in recent decades regarding the formation of ribosomes and the mechanisms and interactions underlying their structure and function. Medline database searches for terms such as “mutant ribosome” or “altered ribosome” demonstrate that specific changes in ribosome structure and function have been described in the art. Such altered ribosomes include ribosomes with altered mRNA binding affinities (Prescott and Goringer, Nucleic Acids Res. 18: 5.381-5386 (1990)), altered co-factor binding affinities (Prescott and Göringer, Nucleic Acids Rec. 18: 5381-5386 (1990)), altered aminoacyl-tRNA binding affinities (Braverman et al., Nucleic Acids Res. 2: 501-507 (1975)), altered peptidyl-tRNA binding affinities (Meskauskas and Dinman, RNA 7: 1084-1096 (2×001)), altered peptide release (Youngman et al., Cell 117: 589-599 (2004)), altered sensitivity to protein synthesis inhibitors (Ono et al., Mol. Cell. Biol. 2: 599-606 (1982)), altered mRNA:tRNA translocation (Southworth et al., J. Mol. Biol. 324: 611-623 (2002)), and altered peptidyltransferase activities (Thompson et al., Proc. Natl. Acad. Sci. 98: 9002-9007 (2001)) as well as ribosomes that read through nonsense (Prescott and Göringer, Nucleic Acids Res. 18: 5381-5386 (1990)) or stop codons (Thompson et al., Proc. Natl. Acad. Sci. 98: 9002-9007 (2001)), ribosomes that recognize mutated mRNA species (Hui and de Boer, Proc. Natl. Acad. Sci. 84: 4762-4766 (1987)), and ribosomes that allow incorporation of nonproteinogenic amino acids into proteins (Dedkova et al., J. Am. Chem. Soc. 125: 661-6617 (2003)).

Over thirty years ago, it was shown that the natural ribosome has catalytic activity for forming ester as well as peptide (amide) bonds (Fahnesock and Rich Science 173:340-343 (1971)). Non-ribosomal biological polypeptide synthesis also plays with this amide/ester similarity in its normal cellular operation. Therefore, in order the construct alternative or unnatural biomimetic structures using cellular machinery, the ribosome is an integral part of this alternative biosynthetic pathway.

Recent articles have expanded on what was previously known of the structure of the ribosome. In prokaryotes, much research has been devoted to both the smaller unit, known by its centrifugal weight as the 30S unit, and to the larger subunit, known as the SOS unit Together the combined intact ribosome is known by its weight as 70S.

In particular the work of Nissen et al. (Science 289: 920-930 (2000)) and Schluenzen et al. (Cell, Vol. 102, 615-623, (2000)), both of which are entirely incorporated by reference, provided new insight on the ribosomal subunits. For the 50S subunit, the Nissen et al indicated that the purpose of the SOS subunit is to “weld” the polypeptide chain together through its catalytic active site. For the smaller 30S subunit, Schluenzen et al article showed the purpose of the 30S subunit is to function as a “reading” unit that ingests the mRNA peptide building instructions and then aligns the tRNA-monomer complexes against the SOS polymerizing site.

Understanding the structure and function of both ribosomal subunits and the peptide synthesis process can be critical to reengineering the natural peptide synthesis system to produce the directed element polymers of the present invention. For example, Nissen et al. observed the chemical symmetry between catalysts that form bonds and the catalysts that break the same bonds. Nissen et al. noted the symmetry between the specific atoms of the active site of the ribosome and very similar individual atoms comprising the active site of a hydrolyzing digestive enzyme such as chymotrypsin that degrades proteins into their constituent amino acids.

3. Biomimetics

Biomimetics is a field in which natural biological structures and functions are mimicked using components or systems not utilized for the same structures and functions by biological organisms. The field embraces computational, mechanical, industrial, and biological applications (Drexler, Proc. Natl. Acad. Sci. 78: 5275-5278 (1981); Cui and Gao, Biotechnol. Prog. 19: 683-692 (2003); Sarikaya et al., Nat. Mater. 2: 577-585 (2003)). Biomimetics is also a hybrid field based on nanotechnology and biotechnology in which products and functions of biological systems can be engineered to interact with inorganic compounds for uses in nanotechnology and biotechnology (See e.g, Sarikaya et al., Nat. Mater. 2: 577-585 (2003)). A benefit of such engineering is that biological systems allow for specific recognition of molecular substrates in catalytic reactions and allow for assembly of structures from a molecular level (Id.).

Biomimetics also encompasses the creation of artificial organisms and new biological systems (i.e., biosystems, also encompassing in vitro or cell-free biological components, products, and functions) (See e.g., Liu and Schultz, Proc. Natl. Acad. Sci. 96: 4780-4785 (1999): Hesman, Sci. News 157: 360 (2000). Such organisms and biosystems produce unnatural products or carry out unnatural functions. The term ‘unnatural’ in this context means not naturally utilized or produced in the mimicked biological processes.

Biomimetic forms of translation have previously utilized modified forms of tRNA (Liu and Schultz, Proc. Natl. Acad. Sci. 96: 4780-4785 (1999)), modified nucleobases (See e.g., Kool, Acc. Chem. Rev. 35: 936-943 (2002)), and modified codon-anticodon pairing (Hohsaka and Sisido, Curr. Opin. Chem. Biol. 4: 645-652 (2002)). In terms of non-biomimetic structures, mutant ribosomes in the art have been utilized to incorporate D-amino acids into proteins (Dedkova et al., J. Am. Chem. Soc. 125: 6616-6617 (2003)). As noted by Drexler, the molecular machinery associated with protein synthesis and function is a powerful tool by which the complex synthetic strategies of conventional organic chemistry can be side-stepped by site-specific synthetic reactions associated with engineered biological mechanisms (Proc. Natl. Acad. Sci. 78: 5275-5278 (1981)).

Modified ribosomes as contemplated by the invention allow for the possibility of a broader range and greater specificity of synthesis than available with current biomimetic products and methods. For example, the ability of molecules to mimic tRNA structure and function within specific ribosomal binding sites offers a means by which an extensive range of biomimetic products can be produced (See e.g, Wilson and Nierhaus, Angew, Chem. Int. Ed, 42: 3464-3486 (2003)).

Bioplastics have been created since the 19^{th} century with early 20^{th} century ventures such as Henry Ford's “Soy Plastic Car.” Much of the current interest is in starch polymers, polysaccharides, proteins, polyhydroxyalkanoates, polylactic acid, and polymers of triglycerides. The rapidly growing polylactate (PLA) biodegradable plastics industry is an example of use of biologically derived monomers. PLA has been jointly developed by Cargill and Dow in a project called NatureWorks® LLC. While this method uses bacteria to convert corn sugar to lactic acid and then produce the monomer substrate lactide, it still requires industrial polymerization techniques to create the polymers NatureWorks® PLA and Ingeo.

The current invention overcomes the problems associated with traditional polymerization processes by providing methods of producing biomimetic structures, such as directed element polymers, using modified ribosomes.

### BRIEF SUMMARY OF THE INVENTION

The present invention is directed to a method of producing a nanotechnological or biomimetic structure comprising:
(a) forming a mixture comprising:
   (i) a modified ribosome,
   (ii) natural or unnatural coding material,
   (iii) natural or unnatural substrate molecules for assembly into a nanotechnological or biomimetic structure, and
   (iv) natural or unnatural factors required for synthesis of the nanotechnological or biomimetic structure during the initiation, elongation, or termination phases of assembly, and
(b) reacting the mixture under conditions capable of producing a nanotechnological or biomimetic structure,
   wherein a nanotechnological or biomimetic structure is produced.

The present invention is also directed to a dual mode in vivo method of producing a nanotechnological or biomimetic structure in a host cell comprising:
(c) forming a mixture comprising:
   (i) a modified ribosome(s),
   (ii) a natural ribosome(s),
   (iii) natural or unnatural coding material,
   (iv) natural or unnatural substrate molecules for assembly into a nanotechnological or biomimetic structure, and
   (v) natural or unnatural factors required for synthesis of the nanotechnological or biomimetic structure during the initiation, elongation, or termination phases of assembly, and
(d) reacting the mixture of step (a) under conditions capable of producing a nanotechnological or biomimetic structure,
   wherein a nanotechnological or biomimetic structure is produced in a host cell.

The invention is also directed to a pedestal mount in vitro method of producing a nanotechnological or biomimetic structure comprising
(a) forming a mixture comprising:
   (i) a modified ribosome possessing a pedestal in the ribosome structure that serves as an acceptor site for a substrate molecule,
   (ii) natural or unnatural coding material,
   (iii) natural or unnatural substrate molecules for assembly into a nanotechnological or biomimetic structure, and
   (iv) natural or unnatural factors required for synthesis of the nanotechnological or biomimetic structure during the initiation, elongation, or termination phases of assembly, and
(b) reacting said mixture of step (a) under conditions capable of producing a nanotechnological or biomimetic structure,
wherein a nanotechnological or biomimetic structure is produced in a host cell.

In some embodiments, the method further comprises the step of isolating the nanotechnological or biomimetic structure.

In some embodiments, the method further comprises the step of mutating the host cell to produce a different nanotechnological or biomimetic structure from the biomimetic or nanotechnological structure produced in a non-mutated host cell.

In some embodiments, the structure produced has at least one dimension on a scale of nanometers.

In some embodiments, the conditions of the methods of producing a nanotechnological or biomimetic structure occur under nonphysiological conditions selected from the group consisting of elevated or reduced pressure, elevated or reduced temperature, elevated or reduced pH, and combinations thereof.

In some embodiments, the unnatural coding material of the mixture comprises modified nucleoside bases or non-nucleoside base replacements.

In some embodiments, the substrate of the mixture comprises natural or unnatural amino acids, modified natural or unnatural amino acids, non-amino acid molecules suitable for assembly into a nanotechnological or biomimetic structure, or combinations thereof. In some embodiments, the substrate of the mixture further comprises a metal.

In some embodiments, the natural or unnatural factors of the mixture further comprise an acceptor molecule selected from the group consisting of natural tRNA, unnatural tRNA, an acceptor molecule capable of interacting with the coding material of the mixture to assemble substrate molecules into a nanotechnological or biomimetic structure, and combinations thereof.

In some embodiments, the acceptor molecule of the mixture interacts with coding material of the mixture in sequences greater or less than three bases or base replacements in length.

In some embodiments, the modified ribosome of the mixture is capable of acting as an acceptor molecule for assembling the substrates of the mixture into a nanotechnological or biomimetic structure.

In some embodiments, the natural or unnatural factors of the mixture comprise natural or unnatural catalysts that transfer substrates to acceptor molecules.

In some embodiments, the reacting of the mixture in step (b) comprises an in vivo, in vitro, or a cell-free system.

In some embodiments, the modified ribosome, natural or unnatural coding material, natural or unnatural factors of the mixture, and combinations thereof are introduced into the mixture using a genetic delivery system. In some embodiments, the genetic delivery system is a virus, plasmid, or other coding material and wherein the conditions are appropriate for expression of the coding material.

The present invention is also directed to a modified ribosome capable of assembling a nanotechnological or biomimetic structure. In some embodiments, the ribosome is modified from a natural ribosome.

In some embodiments, the modified ribosome comprises ribosomal subunits in natural or unnatural combinations. In some embodiments, the ribosomal subunits comprise natural or unnatural mixtures.

In some embodiments, the modified ribosome comprises an unnatural molecular size, molecular weight, or combination thereof.

The present invention is also directed to a method of modifying a ribosome, the method comprising:
(a) observing the degradative process used by hydrolyzing enzymes to break a chemical bond; and
(b) modifying the active site of a natural ribosome to produce the reverse reaction of the observed degradative process.

In some embodiments, the reverse reaction is designed to synthesize cellulose. In some embodiments, the reverse reaction is designed to synthesize polylactate.

The present invention is also directed to an unnatural acceptor molecule comprising a molecule capable of transporting a substrate to a modified ribosome. In some embodiments, the unnatural acceptor molecule is an unnatural tRNA.

In some embodiments, the codon sequence length exceeds three nucleotides but is less than ten nucleotides in length.

The present invention is also directed to the nanotechnological or biomimetic structure produced using any of the methods of the present invention. In some embodiments, the biomimetic structure a polymer or macrocyclic molecule.

The present invention is also directed to a nonhuman organism comprising a modified ribosome, wherein the nonhuman organism is capable of synthesizing a nanotechnological or biomimetic structure.

The present invention is also directed to a host cell comprising a modified ribosome, wherein the host cell is capable of synthesizing a nanotechnological or biomimetic structure.

The present invention is also directed to a method of producing a nanotechnological or biomimetic structure in a host cell comprising:
(a) forming a mixture comprising:
   (i) a modified ribosome(s),
   (ii) a natural ribosome(s),
   (iii) natural or unnatural coding material,
   (iv) natural or unnatural substrate molecules for assembly into a nanotechnological or biomimetic structure, and
   (v) natural or unnatural factors required for synthesis of the nanotechnological or biomimetic structure during the initiation, elongation, or termination phases of assembly, and
(b) reacting the mixture of step (a) under conditions capable of producing a nanotechnological or biomimetic structure,
(c) isolating the nanotechnological or biomimetic structure by storing, secreting or directly secreting the nanotechnological or biomimetic structure,
   wherein a nanotechnological or biomimetic structure is produced in a host cell. In some embodiments, the isolation is performed by temporal or spatial isolation. In further embodiments, the isolation method comprises modifying signal recognition protein (SRP) carriers, modifying chaperone proteins or modifying cellular translocons. In another embodiment, the invention further comprising the steps of mutating the parallel dual mode in vivo pathway of the host cell to produce a different nanotechnological or biomimetic structure from the biomimetic or nanotechnological structure produced in a non-mutated host cell. In some embodiments, the modified ribosomes are attached to a membrane or is free-floating within the cell.

In some embodiments, spatial isolation comprises separation by enclosure in a membraneous structure. In further embodiments, the membraneous structure is a lipid bilayer

### BRIEF DESCRIPTION OF THE FIGURES

FIGS. 1A and B illustrate in a 3-dimensional graph the possibility space for creating the directed element polymers of the present invention (FIG. 1A) and open-ended class of possible polymers (FIG. 1B).

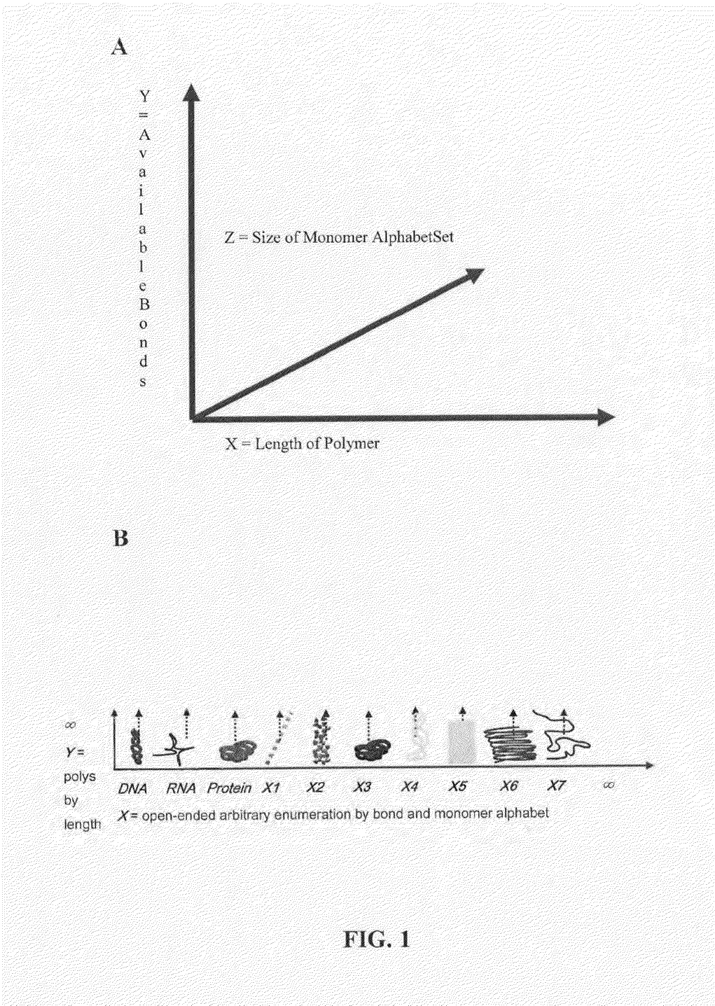

FIG. 2 illustrates the natural process for synthesizing proteins using a natural ribosome.

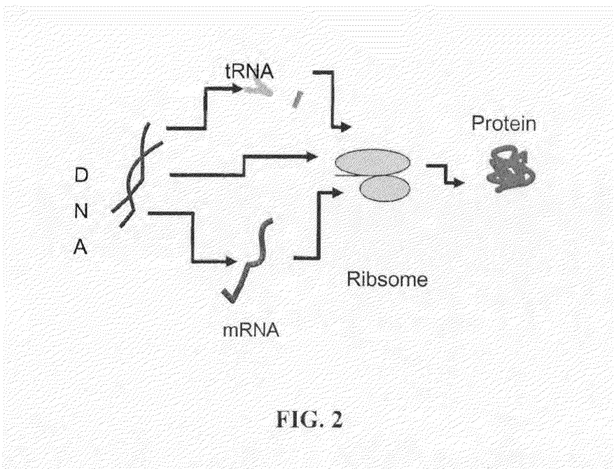

FIG. 3 illustrates an exemplary method, the dual mode in vivo method, of synthesizing a directed element polymer using the present invention.

FIG. 4 illustrates the Temporal Mode In Vivo (TMIV) isolation method.

FIG. 5 illustrates the Isolated Mode In Vivo (IMIV) isolation method.

FIG. 6 illustrates the Autologous Mode In Vivo (AMIV) synthesis method.

FIG. 7 illustrates the Multiple Mode In Vivo (MMIV) synthesis method.

### DETAILED DESCRIPTION OF THE INVENTION

This invention is directed to modifying a natural ribosome to produce new or existing copolymers, each based on a chemical bond other than the peptide bond, but as with natural proteins, each sequenced by design from a template. These copolymers constructed from a set of monomers using a chemical bond other than a peptide bond are called Directed Element Polymers (DEPs). In some embodiments, the DEPs will show all the versatility of use that characterize proteins. However, these polymers are not produced by industrial means, Instead modified living systems produce the polymers using a method referred to as a Biological Industrial Operational Polymer (BIOP) paradigm.

The terms “unnatural”, “alternative” and “modified” polymers are used interchangeably herein.

As described in the sections to follow, the present invention is directed to a novel method of producing directed element polymers using living systems comprising a modified ribosome, novel copolymers known as directed element polymers, and methods of using the same. The section headings below are for organizational purposes only and are not intended to impart any division or meaning to this document unless specified otherwise.

### The BIOP Method

This invention is directed to modified ribosomes capable of assembling biomimetic structures. A biomimetic structure as contemplated by the invention is an unnatural polymer or structure other than a polymer consisting solely of naturally occurring amino acids. As used in this application, this unnatural polymer or structure shall also be referred to as a directed element polymer (DEP) which comprises directed elements. Based on this definition, the mixed amino acid polymer produced by a mutant ribosome as described in Dedkova et al. (J. Am. Chem. Soc. 125: 6616-6617 (2003)) would not be encompassed by the invention. In fact such polymers consisting solely of mixed D and L amino acids are considered proteins rather than biomimetic structures, given that natural proteins may contain D-amino acids as a result of posttranslational modification or nonribosomal synthesis (See Hohsaka and Sisido, Curr. Opin. Chem. Biol. 4: 645-652 (2002): Dedkova et al., J. Am. Chem. Soc. 125: 6616-6617 (2003)).

The term “about” when used in conjunction with a percentage or other numerical amount means plus or minus 10% of that percentage or other numerical amount. For example, the term “about 80%” would encompass 80% plus or minus 8%.

The term “modified” as used herein means that the item being modified has been changed in form or character. For example, a modified ribosome is a natural ribosome which has been changed in form or character, e.g., so that it can synthesis a DEP.

The term “substrate” refers to a substance which is acted upon. For example a substrate can be the substance upon which an enzyme acts.

The term “coding material” refers to any substance which can be used to record the order with which a substrate will be inserted into a directed element polymer and wherein the substance can function as a template for directing synthesis of a directed element polymer. For example, in some embodiments, the coding material is a nucleic acid sequence. The coding material of the present invention may itself serve as the template for the synthesis of the directed element polymer, e.g., mRNA, or it may require transcription before acting as the template for synthesis, e.g. DNA being transcribed into mRNA.

Insertion of Coding Material into a Host Cell

In some embodiments, the BIOP method begins with the insertion of coding material into a host cell. Any insertion method known to one of skill in the art can be used, for example, propagating the coding material in the host cell using a vector. Suitable techniques for the present invention, for example, are disclosed in Molecular Biology of the Gene, 5th ed. (Cold Spring Harbor Laboratory Press 2004) herein incorporated by reference in its entirety.

In some embodiments, the coding material is a nucleic acid sequence. These nucleic acids, either DNA or RNA, can be inserted to provide the template for synthesizing the desired DEP. Similar to traditional cellular function, the nucleic acid will contain the information needed to correctly sequence the directed elements into a polymer. For example, if DNA is used, the mRNA produced from this DNA sequence can be used by the modified ribosome to determine the order in which each substrate, such as a directed element, is inserted into the DEP. If RNA is used, then the inserted nucleic acid can be the template used to order the substrates, such as directed elements, within the DEP.

In some embodiments, the coding material is inserted into a nonhuman organism comprising a modified ribosome. In some embodiments, the nonhuman organism is selected from the group consisting of a virus, plasmid, bacteria, fungi, or nonhuman eukaryotic cell. In some embodiments, the nonhuman organism comprising a modified ribosome is capable of synthesizing a nanotechnological or biomimetic structure.

In some embodiments, the coding material is inserted into a host cell comprising a modified ribosome. In some embodiments, the host cell is E. coli. In some embodiments, the host cell comprising a modified ribosome is capable of synthesizing a nanotechnological or biomimetic structure.

Attachment of a Substrate to a tRNA

Natural tRNAs are charged by the attachment of an amino acid to the 3′ terminal adenosine nucleotide via a high-energy acyl linkage as described in Molecular Biology of the Gene. In contrast, those tRNAs without an attached amino acid are considered uncharged. The later hydrolysis of the acyl linkage results in a large change in free energy. This energy is released when the bond is broken and is used to help drive the formation of peptide bonds which link amino acids to each other in polypeptide chains.

In some embodiments, the present invention uses a high-energy bond to attach a substrate molecule to a tRNA. In some embodiments, the present invention uses a high-energy acyl linkage to attach a substrate molecule to a tRNA. In some embodiments, the subsequent breakage of the high-energy bond attaching the substrate to the tRNA is used to aid in the formation of the bonds between the substrate molecules used to form a directed element polymer. As one of skill in the art can appreciate, any suitable high-energy bond which can link the desired substrate to the tRNA can be used in the present invention. Suitable bonds can be determined by examining the chemical bonding properties of both the substrate molecule and the tRNA. e.g., to determine the appropriate chemical groups on each substrate to be linked and the proper chemical bond used to link the identified chemical groups.

In some embodiments, the present invention uses a low-energy bond or multiple low energy-bonds to attach a substrate molecule to a tRNA. In some embodiments, the subsequent breakage of the low-energy bond attaching the substrate to the tRNA is used to aid in the formation of the bonds between the substrate molecules used to form a directed element polymer. In some embodiments, the subsequent breakage of multiple low-energy bonds attaching the substrate to the tRNA is used to aid in the formation of the bonds between the substrate molecules to form a directed element polymer. As one of skill in the art can appreciate, any suitable low-energy bond, or combination of low-energy bonds, which can link the desired substrate to the tRNA can be used in the present invention. Suitable bonds can be determined by examining the chemical bonding properties of both the substrate molecule and the tRNA, e.g., to determine the appropriate chemical groups on each substrate to be linked and the proper chemical bond used to link the identified chemical groups.

In some embodiments, the present invention uses natural or modified tRNA synthetases to attach a substrate molecule to a tRNA, unnatural tRNA, or other acceptor molecule. In natural peptide synthesis, each of the twenty amino acids is attached to the appropriate tRNA by a single, dedicated tRNA synthetase. A single tRNA synthetase, while limited to attaching only one amino acid, can attach this amino acid to any number of tRNAs, each of which is responsible for the amino acid. The present invention, in some embodiments, uses a modified tRNA synthetase or other molecule capable of mimicking the function of tRNA, to attach the substrate molecule to the modified or unnatural tRNA used in the BIOP method.

Unnatural tRNA

To produce a biomimetic polymer like a directed element polymer in a modified ribosome, an unnatural acceptor molecule, such as a modified tRNA, must bring the desired substrate to the active site of the ribosome. A natural tRNA molecule is covalently linked to a single amino acid. Using the anticodon, which is complementary to the codon in the mRNA representing the amino acid, the (RNA delivers its amino acid to the active site of the natural ribosome for insertion into the elongating polymer at the correct location.

The present invention uses these concepts to engineer a modified tRNA, or other molecule capable of mimicking the function of tRNA, to deliver non-amino acid substrates from the cytosol to the active site of the modified ribosome. These non-amino acid substrates are then incorporated into the elongating directed element polymer at a location determined by the anti-codon, or similar localizing mechanism, on the modified tRNA or other molecule capable of mimicking the function of tRNA. It is contemplated that elongation of the claimed biomimetic polymers by modified ribosomes can occur in an ordered sequence that mimics the ordered sequence by which amino acids are assembled during natural translation. For in vivo systems, this modified tRNA or other molecule can be designed so that a natural ribosome can not recognize it.

In some embodiments, the anti-codon on the unnatural tRNA is of an unnatural length. The traditional anti-codon is three nucleotides in length and pairs with a codon of equal length. To provide a successful dual mode in vivo system, it may be necessary to engineer codons of length greater than the natural three nucleotide sequences to prevent interference between the BIOP systems and the natural mechanisms which sustain cellular function.

In some embodiments, the anti-codon of the unnatural tRNA has a nucleotide sequence which exceeds 3 nucleotides in length. In some embodiments, the anti-codon of the unnatural (RNA is between about 3 to about 20 nucleotides in length. In some embodiments, the anti-codon of the unnatural tRNA is between about 3 to about 10 nucleotides in length. In some embodiments, the anti-codon of the unnatural tRNA is between about 3 to about 5 nucleotides in length.

As one of skill in the art will recognize, in the embodiments of the present invention where the anti-codon of the unnatural tRNA is of a length longer than 3 nucleotides, the codon sequence found on the mRNA will be of equal length to preserve the complementarity of the two components. Therefore, in some embodiments, the codon has a nucleotide sequence which exceeds 3 nucleotides in length. In some embodiments, the codon between about 3 to about 20 nucleotides in length. In some embodiments, the codon is between about 3 to about 10 nucleotides in length. In some embodiments, the codon is between about 3 to about 5 nucleotides in length.

### Modified Ribosomes

The natural ribosome is the essential manufacturing engine of all living cells, Ribosomes read information and assemble and output manufactured substance. Their input is the sequential instructions from messenger ribonucleic acid (mRNA). Their products are sequenced polymerized strings of amino acids where the amino acid molecules are bonded with a peptide bond. These copolymers are proteins and are an example of a naturally occurring DEP.

In some embodiments, the present invention comprises a modified ribosome produced by modification of a natural ribosome. Such modification may be induced using any method of genetic engineering available to one skilled in the art, including but not limited to, mutagenesis and selective screening techniques.

In some embodiments, the A, E, and P sites of a natural ribosome can be modified to accommodate the binding of the unnatural tRNAs of the present invention. These sites are the natural binding sites for aminoacylated-tRNA (A site), peptidyl-tRNA (P site), and tRNAs which have been released after the growing polypeptide chain has been transferred to the aminoacyl-tRNA (E site). These sites can be altered to accommodate the differences in molecular size or primary, secondary, tertiary, or quaternary chemical structure of both the unnatural RNA and its substrate when compared to a natural tRNA carrying its substrate, for example, an amino acid.

A natural ribosome may be modified structurally based on the requirements needed to form the desired chemical bond. The ribosomal active site may be modified at either the large or small ribosomal component or at both subunits within the present invention. For example, in prokaryotes the active site modifications may be made on either the SOS or 30S subunit. Similarly, in eukaryotes, the active site modifications may be made on either the 60S or 40S subunit. In particular, modifications can be necessary on the 30S subunit, so that only those mRNA instructions intended for use in the modified parallel synthesis system, i.e. the synthesis system present in an otherwise natural cell which forms DEPs, are used in the parallel synthesis system not the natural system. Further, the 50S active site can be modified to allow for the polymerization of novel monomers.

A natural ribosomal active site, for example, the adenosine base on the 23SrRNA in position 2451 on E. coli. can be modified to synthesize the desired bond in the DEP. Because this base is normally conserved, except for position changes, in most life forms (except a few archaebacteria) it appears to be a critical catalyst for ribosomal synthesis reactions by using one of its nitrogen atoms to transfer protons.

The modified ribosome contemplated by the invention includes any combination of ribosomal subunits whether they exist naturally or not. Such subunits may be comprised of unnatural mixtures of proteins, ribosomal RNAs, and other components and may be of molecular sizes or weights that vary from natural subunits.

In some embodiments, the subunits can be modified to alter the ribosomal tunnel and support structure to accommodate bonds with different stiffness, or the tRNA/substrate alignment mechanism in the ribosome to accommodate alternative tRNA's bringing in alternative substrates. For example, the exit tunnel in a natural ribosome can be altered in molecular size and chemical structure to allow unnatural substrates with different molecular sizes, chemical bonds, or chemical binding affinities to pass through the ribosome as they are inserted into a growing DEP.

In some embodiments, this invention is also directed to modified ribosomes with an active site capable of inserting a directed element into a directed element polymer using any known chemical bond. This active site can be designed to synthesize a specific chemical bond by using the symmetry between the reactions for bond synthesis and bond degradation. For example, researchers have noted that there is symmetry between peptide bond synthesis and the acylation step used by serine proteases to degrade these bonds. (Nissen et al., Science 289:920-930 (2000)).

In some embodiments, this invention is directed to examining known enzymatic reactions. e.g., bond degradative reactions, to design a reaction capable of synthesizing the desired chemical bond between a new directed element and the directed element polymer using a modified ribosome. Using this knowledge about the enzymes, e.g, hydrolyzing enzymes or other degradative enzymes, to gain insight on how to effect changes to the catalytic active site of the ribosome is Reverse Reverse Engineering (RRE).

Life uses enzymes to break up other biological polymers. Conversely, except for proteins being constructed using ribosomes, the other biological polymers are constructed using protein enzymes. Examining these paired mechanisms for construction and destruction of natural polymers can be used to determine the ribosomal active site molecules that are required to form the desired chemical bond in the DEP.

This invention contemplates using all known enzymatic reactions as a model for producing a synthesis reaction. Some specific examples include, but are not limited to, using the reactions of reductases, transferases, hydrolases, lyases, isomerases, ligases, amylases, peptidases, and lipases to engineer a synthesis reaction.

Of particular interest, are the hydrolases, examples of which are contained in Table I below. The enzyme number listed below corresponds to the Nomenclature Committee of the International Union of Biochemistry and Molecular Biology (NC-IUBMB) system for numbering enzymes.

TABLE 1
Hydrolases

| Enzyme | |
|---|---|
| Number | |
| (NC-IUBMB | |
| system) | Enzyme Name |
| EC 3.1 | Acting on Ester Bonds |
| EC 3.1.1 | Carboxylic Ester Hydrolases |
| EC 3.1.1.1 | carboxylesterase |
| EC 3.1.1.2 | arylesterase |
| EC 3.1.1.3 | triacylglycerol lipase |
| EC 3.1.1.4 | phospholipase A2 |
| EC 3.1.1.5 | lysophospholipase |
| EC 3.1.1.6 | acetylesterase |
| EC 3.1.1.7 | acetylcholinesterase |
| EC 3.1.1.8 | cholinesterase |
| EC 3.1.1.10 | tropinesterase |
| EC 3.1.1.11 | pectinesterase |
| EC 3.1.1.13 | sterol esterase |
| EC 3.1.1.14 | chlorophyllase |
| EC 3.1.1.15 | L-arabinonolactonase |
| EC 3.1.1.17 | gluconolactonase |
| EC 3.1.1.19 | uronolactonase |
| EC 3.1.1.20 | tannase |
| EC 3.1.1.21 | retinyl-palmitate esterase |
| EC 3.1.1.22 | hydroxybutyrate-dimer hydrolase |
| EC 3.1.1.23 | acylglycerol lipase |
| EC 3.1.1.24 | 3-oxoadipate enol-lactonase |
| EC 3.1.1.25 | 1,4-lactonase |
| EC 3.1.1.26 | galactolipase |
| EC 3.1.1.27 | 4-pyridoxolactonase |
| EC 3.1.1.28 | acylcarnitine hydrolase |
| EC 3.1.1.29 | aminoacyl-tRNA hydrolase |
| EC 3.1.1.30 | D-arabinonolactonase |
| EC 3.1.1.31 | 6-phosphogluconolactonase |
| EC 3.1.1.32 | phospholipase A1 |
| EC 3.1.1.33 | 6-acetylglucose deacetylase |
| EC 3.1.1.34 | lipoprotein lipase |
| EC 3.1.1.35 | dihydrocoumarin hydrolase |
| EC 3.1.1.36 | limonin-D-ring-lactonase |
| EC 3.1.1.37 | steroid-lactonase |
| EC 3.1.1.38 | triacetate-lactonase |
| EC 3.1.1.39 | actinomycin lactonase |
| EC 3.1.1.40 | orsellinate-depside hydrolase |
| EC 3.1.1.41 | cephalosporin-C deacetylase |
| EC 3.1.1.42 | chlorogenate hydrolase |
| EC 3.1.1.43 | a-amino-acid esterase |
| EC 3.1.1.44 | 4-methyloxaloacetate esterase |
| EC 3.1.1.45 | carboxymethylenebutenolidase |
| EC 3.1.1.46 | deoxylimonate A-ring-lactonase |
| EC 3.1.1.47 | 1-alkyl-2-acetylglycerophosphocholine esterase |
| EC 3.1.1.48 | fusarinine-C ornithinesterase |
| EC 3.1.1.49 | sinapine esterase |
| EC 3.1.1.50 | wax-ester hydrolase |
| EC 3.1.1.51 | phorbol-diester hydrolase |
| EC 3.1.1.52 | phosphatidylinositol deacylase |
| EC 3.1.1.53 | sialate O-acetylesterase |
| EC 3.1.1.54 | acetoxybutynylbithiophene deacetylase |
| EC 3.1.1.55 | acetylsalicylate deacetylase |
| EC 3.1.1.56 | methylumbelliferyl-acetate deacetylase |
| EC 3.1.1.57 | 2-pyrone-4,6-dicarboxylate lactonase |
| EC 3.1.1.58 | N-acetylgalactosaminoglycan deacetylase |
| EC 3.1.1.59 | juvenile-hormone esterase |
| EC 3.1.1.60 | bis(2-ethylhexyl)phthalate esterase |
| EC 3.1.1.61 | protein-glutamate methylesterase |
| EC 3.1.1.63 | 11-cis-retinyl-palmitate hydrolase |
| EC 3.1.1.64 | all-trans-retinyl-palmitate hydrolase |
| EC 3.1.1.65 | L-rhamnono-1,4-lactonase |
| EC 3.1.1.66 | 5-(3,4-diacetoxybut-1-ynyl)-2,2′-bithiophene deacetylase |
| EC 3.1.1.67 | fatty-acyl-ethyl-ester synthase |
| EC 3.1.1.68 | xylono-1,4-lactonase |
| EC 3.1.1.70 | cetraxate benzylesterase |
| EC 3.1.1.71 | acetylalkylglycerol acetylhydrolase |
| EC 3.1.1.72 | acetylxylan esterase |
| EC 3.1.1.73 | feruloyl esterase |
| EC 3.1.1.74 | cutinase |
| EC 3.1.1.75 | poly(3-hydroxybutyrate) depolymerase |
| EC 3.1.1.76 | poly(3-hydroxyoctanoate) depolymerase acyloxyacyl |
| | hydrolase |
| EC 3.1.1.77 | acyloxyacyl hydrolase |
| EC 3.1.1.78 | polyneuridine-aldehyde esterase |
| EC 3.1.1.79 | hormone-sensitive lipase |
| EC 3.1.2 | Thiolester Hydrolases |
| EC 3.1.2.1 | acetyl-CoA hydrolase |
| EC 3.1.2.2 | palmitoyl-CoA hydrolase |
| EC 3.1.2.3 | succinyl-CoA hydrolase |
| EC 3.1.2.4 | 3-hydroxyisobutyryl-CoA hydrolase |
| EC 3.1.2.5 | hydroxymethylglutaryl-CoA hydrolase |
| EC 3.1.2.6 | hydroxyacylglutathione hydrolase |
| EC 3.1.2.7 | glutathione thiolesterase |
| EC 3.1.2.10 | formyl-CoA hydrolase |
| EC 3.1.2.11 | acetoacetyl-CoA hydrolase |
| EC 3.1.2.12 | S-formylglutathione hydrolase |
| EC 3.1.2.13 | S-succinylglutathione hydrolase |
| EC 3.1.2.14 | oleoyl-[acyl-carrier-protein] hydrolase |
| EC 3.1.2.15 | ubiquitin thiolesterase |
| EC 3.1.2.16 | [citrate-(pro-3S)-lyase] thiolesterase |
| EC 3.1.2.17 | (S)-methylmalonyl-CoA hydrolase |
| EC 3.1.2.18 | ADP-dependent short-chain-acyl-CoA hydrolase |
| EC 3.1.2.19 | ADP-dependent medium-chain-acyl-CoA hydrolase |
| EC 3.1.2.20 | acyl-CoA hydrolase |
| EC 3.1.2.21 | dodecanoyl-[acyl-carrier protein] hydrolase |
| EC 3.1.2.22 | palmitoyl[protein] hydrolase |
| EC 3.1.2.23 | 4-hydroxybenzoyl-CoA thioesterase |
| EC 3.1.2.24 | 2-(2-hydroxyphenyl)benzenesulfinate hydrolase |
| EC 3.1.2.25 | phenylacetyl-CoA hydrolase |
| EC 3.1.3 | Phosphoric Monoester Hydrolases |
| EC 3.1.3.1 | alkaline phosphatase |
| EC 3.1.3.2 | acid phosphatase |
| EC 3.1.3.3 | phosphoserine phosphatase |
| EC 3.1.3.4 | phosphatidate phosphatase |
| EC 3.1.3.5 | 5′-nucleotidase |
| EC 3.1.3.6 | 3′-nucleotidase |
| EC 3.1.3.7 | 3′(2′),5′-bisphosphate nucleotidase |
| EC 3.1.3.8 | 3-phytase |
| EC 3.1.3.9 | glucose-6-phosphatase |
| EC 3.1.3.10 | glucose-1-phosphatase |
| EC 3.1.3.11 | fructose-bisphosphatase |
| EC 3.1.3.12 | trehalose-phosphatase |
| EC 3.1.3.13 | bisphosphoglycerate phosphatase |
| EC 3.1.3.14 | methylphosphothioglycerate phosphatase |
| EC 3.1.3.15 | histidinol-phosphatase |
| EC 3.1.3.16 | phosphoprotein phosphatase |
| EC 3.1.3.17 | [phosphorylase] phosphatase |
| EC 3.1.3.18 | phosphoglycolate phosphatase |
| EC 3.1.3.19 | glycerol-2-phosphatase |
| EC 3.1.3.20 | phosphoglycerate phosphatase |
| EC 3.1.3.21 | glycerol-1-phosphatase |
| EC 3.1.3.22 | mannitol-1-phosphatase |
| EC 3.1.3.23 | sugar-phosphatase |
| EC 3.1.3.24 | sucrose-phosphatase |
| EC 3.1.3.25 | inositol-phosphate phosphatase |
| EC 3.1.3.26 | 4-phytase |
| EC 3.1.3.27 | phosphatidylglycerophosphatase |
| EC 3.1.3.28 | ADPphosphoglycerate phosphatase |
| EC 3.1.3.29 | N-acylneuraminate-9-phosphatase |
| EC 3.1.3.31 | nucleotidase |
| EC 3.1.3.32 | polynucleotide 3′-phosphatase |
| EC 3.1.3.33 | polynucleotide 5′-phosphatase |
| EC 3.1.3.34 | deoxynucleotide 3′-phosphatase |
| EC 3.1.3.35 | thymidylate 5′-phosphatase |
| EC 3.1.3.36 | phosphoinositide 5-phosphatase |
| EC 3.1.3.37 | sedoheptulose-bisphosphatase |
| EC 3.1.3.38 | 3-phosphoglycerate phosphatase |
| EC 3.1.3.39 | streptomycin-6-phosphatase |
| EC 3.1.3.40 | guanidinodeoxy-scyllo-inositol-4-phosphatase |
| EC 3.1.3.41 | 4-nitrophenylphosphatase |
| EC 3.1.3.42 | [glycogen-synthase-D] phosphatase |
| EC 3.1.3.43 | [pyruvate dehydrogenase (lipoamide)]-phosphatase |
| EC 3.1.3.44 | [acetyl-CoA carboxylase]-phosphatase |
| EC 3.1.3.45 | 3-deoxy-manno-octulosonate-8-phosphatase |
| EC 3.1.3.46 | fructose-2,6-bisphosphate 2-phosphatase |
| EC 3.1.3.47 | [hydroxymethylglutaryl-CoA reductase (NADPH)]- |
| | phosphatase |
| EC 3.1.3.48 | protein-tyrosine-phosphatase |
| EC 3.1.3.49 | [pyruvate kinase]-phosphatase |
| EC 3.1.3.50 | sorbitol-6-phosphatase |
| EC 3.1.3.51 | dolichyl-phosphatase |
| EC 3.1.3.52 | [3-methyl-2-oxobutanoate dehydrogenase (lipoamide)]- |
| | phosphatase |
| EC 3.1.3.53 | [myosin-light-chain] phosphatase |
| EC 3.1.3.54 | fructose-2,6-bisphosphate 6-phosphatase |
| EC 3.1.3.55 | caldesmon-phosphatase |
| EC 3.1.3.56 | inositol-polyphosphate 5-phosphatase |
| EC 3.1.3.57 | inositol-1,4-bisphosphate 1-phosphatase |
| EC 3.1.3.58 | sugar-terminal-phosphatase |
| EC 3.1.3.59 | alkylacetylglycerophosphatase |
| EC 3.1.3.60 | phosphoenolpyruvate phosphatase |
| EC 3.1.3.62 | multiple inositol-polyphosphate phosphatase |
| EC 3.1.3.63 | 2-carboxy-D-arabinitol-1-phosphatase |
| EC 3.1.3.64 | phosphatidylinositol-3-phosphatase |
| EC 3.1.3.66 | phosphatidylinositol-3,4-bisphosphate 4-phosphatase |
| EC 3.1.3.67 | phosphatidylinositol-3,4,5-trisphosphate 3-phosphatase |
| EC 3.1.3.68 | 2-deoxyglucose-6-phosphatase |
| EC 3.1.3.69 | glucosylglycerol 3-phosphatase |
| EC 3.1.3.70 | mannosyl-3-phosphoglycerate phosphatase |
| EC 3.1.3.71 | 2-phosphosulfolactate phosphatase |
| EC 3.1.3.72 | 5-phytase |
| EC 3.1.3.73 | a-ribazole phosphatase |
| EC 3.1.3.74 | pyridoxal phosphatase |
| EC 3.1.3.75 | phosphoethanolamine/phosphocholine phosphatase |
| EC 3.1.4 | Phosphoric Diester Hydrolases |
| EC 3.1.4.1 | phosphodiesterase I |
| EC 3.1.4.2 | glycerophosphocholine phosphodiesterase |
| EC 3.1.4.3 | phospholipase C |
| EC 3.1.4.4 | phospholipase D |
| EC 3.1.4.11 | phosphoinositide phospholipase C |
| EC 3.1.4.12 | sphingomyelin phosphodiesterase |
| EC 3.1.4.13 | serine-ethanolaminephosphate phosphodiesterase |
| EC 3.1.4.14 | [acyl-carrier-protein] phosphodiesterase |
| EC 3.1.4.15 | adenylyl-[glutamate#% G-#%@ammonia ligase] |
| | hydrolase |
| EC 3.1.4.16 | 2′,3′-cyclic-nucleotide 2′-phosphodiesterase |
| EC 3.1.4.17 | 3′,5′-cyclic-nucleotide phosphodiesterase |
| EC 3.1.4.35 | 3′,5′-cyclic-GMP phosphodiesterase |
| EC 3.1.4.37 | 2′,3′-cyclic-nucleotide 3′-phosphodiesterase |
| EC 3.1.4.38 | glycerophosphocholine cholinephosphodiesterase |
| EC 3.1.4.39 | alkylglycerophosphoethanolamine phosphodiesterase |
| EC 3.1.4.40 | CMP-N-acylneuraminate phosphodiesterase |
| EC 3.1.4.41 | sphingomyelin phosphodiesterase D |
| EC 3.1.4.42 | glycerol-1,2-cyclic-phosphate 2-phosphodiesterase |
| EC 3.1.4.43 | glycerophosphoinositol inositolphosphodiesterase |
| EC 3.1.4.44 | glycerophosphoinositol glycerophosphodiesterase |
| EC 3.1.4.45 | N-acetylglucosamine-1-phosphodiester a-N- |
| | acetylglucosaminidase |
| EC 3.1.4.46 | glycerophosphodiester phosphodiesterase |
| EC 3.1.4.48 | dolichylphosphate-glucose phosphodiesterase |
| EC 3.1.4.49 | dolichylphosphate-mannose phosphodiesterase |
| EC 3.1.4.50 | glycosylphosphatidylinositol phospholipase D |
| EC 3.1.4.51 | glucose-1-phospho-D-mannosylglycoprotein |
| | phosphodiesterase |
| EC 3.1.5 | Triphosphoric Monoester Hydrolases |
| EC 3.1.5.1 | dGTPase |
| EC 3.1.6 | Sulfuric Ester Hydrolases |
| EC 3.1.6.1 | arylsulfatase |
| EC 3.1.6.2 | steryl-sulfatase |
| EC 3.1.6.3 | glycosulfatase |
| EC 3.1.6.4 | N-acetylgalactosamine-6-sulfatase |
| EC 3.1.6.6 | choline-sulfatase |
| EC 3.1.6.7 | cellulose-polysulfatase |
| EC 3.1.6.8 | cerebroside-sulfatase |
| EC 3.1.6.9 | chondro-4-sulfatase |
| EC 3.1.6.10 | chondro-6-sulfatase |
| EC 3.1.6.11 | disulfoglucosamine-6-sulfatase |
| EC 3.1.6.12 | N-acetylgalactosamine-4-sulfatase |
| EC 3.1.6.13 | iduronate-2-sulfatase |
| EC 3.1.6.14 | N-acetylglucosamine-6-sulfatase |
| EC 3.1.6.15 | N-sulfoglucosamine-3-sulfatase |
| EC 3.1.6.16 | monomethyl-sulfatase |
| EC 3.1.6.17 | D-lactate-2-sulfatase |
| EC 3.1.6.18 | glucuronate-2-sulfatase |
| EC 3.1.7 | Diphosphoric Monoester Hydrolases |
| EC 3.1.7.1 | prenyl-diphosphatase |
| EC 3.1.7.2 | guanosine-3′,5′-bis(diphosphate) 3′-diphosphatase |
| EC 3.1.7.3 | monoterpenyl-diphosphatase |
| EC 3.1.8 | Phosphoric Triester Hydrolases |
| EC 3.1.8.1 | aryldialkylphosphatase |
| EC 3.1.8.2 | diisopropyl-fluorophosphatase |
| EC 3.1.11 | Exodeoxyribonucleases Producing 5′-Phosphomonoesters |
| EC 3.1.11.1 | exodeoxyribonuclease I |
| EC 3.1.11.2 | exodeoxyribonuclease III |
| EC 3.1.11.3 | exodeoxyribonuclease (lambda-induced) |
| EC 3.1.11.4 | exodeoxyribonuclease (phage SP3-induced) |
| EC 3.1.11.5 | exodeoxyribonuclease V |
| EC 3.1.11.6 | exodeoxyribonuclease VII |
| EC 3.1.13 | Exoribonucleases Producing 5′-Phosphomonoesters |
| EC 3.1.13.1 | exoribonuclease II |
| EC 3.1.13.2 | exoribonuclease H |
| EC 3.1.13.3 | oligonucleotidase |
| EC 3.1.13.4 | poly(A)-specific ribonuclease |
| EC 3.1.14 | Exoribonucleases Producing 3′-Phosphomonoesters |
| EC 3.1.14.1 | yeast ribonuclease |
| EC 3.1.15 | Exonucleases Active with either Ribo- or |
| | Deoxyribonucleic Acids and Producing 5′- |
| | Phosphomonoesters |
| EC 3.1.15.1 | venom exonuclease |
| EC 3.1.16 | Exonucleases Active with either Ribo- or |
| | Deoxyribonucleic Acids and Producing 3′- |
| | Phosphomonoesters |
| EC 3.1.16.1 | spleen exonuclease |
| EC 3.1.21 | Endodeoxyribonucleases Producing 5′- |
| | Phosphomonoesters |
| EC 3.1.21.1 | deoxyribonuclease I |
| EC 3.1.21.2 | deoxyribonuclease IV (phage-T4-induced) |
| EC 3.1.21.3 | type I site-specific deoxyribonuclease |
| EC 3.1.21.4 | type II site-specific deoxyribonuclease |
| EC 3.1.21.5 | type III site-specific deoxyribonuclease |
| EC 3.1.21.6 | CC-preferring endodeoxyribonuclease |
| EC 3.1.21.7 | deoxyribonuclease V |
| EC 3.1.22 | Endodeoxyribonucleases Producing 3′- |
| | Phosphomonoesters |
| EC 3.1.22.1 | deoxyribonuclease II |
| EC 3.1.22.2 | Aspergillus deoxyribonuclease K1 |
| EC 3.1.22.4 | crossover junction endodeoxyribonuclease |
| EC 3.1.22.5 | deoxyribonuclease X |
| EC 3.1.25 | Site-Specific Endodeoxyribonucleases Specific for |
| | Altered Bases |
| EC 3.1.25.1 | deoxyribonuclease (pyrimidine dimer) |
| EC 3.1.26 | Endoribonucleases Producing 5′-Phosphomonoesters |
| EC 3.1.26.1 | Physarum polycephalum ribonuclease |
| EC 3.1.26.2 | ribonuclease alpha |
| EC 3.1.26.3 | ribonuclease III |
| EC 3.1.26.4 | calf thymus ribonuclease H |
| EC 3.1.26.5 | ribonuclease P |
| EC 3.1.26.6 | ribonuclease IV |
| EC 3.1.26.7 | ribonuclease P4 |
| EC 3.1.26.8 | ribonuclease M5 |
| EC 3.1.26.9 | ribonuclease [poly-(U)-specific] |
| EC 3.1.26.10 | ribonuclease IX |
| EC 3.1.26.11 | tRNase Z |
| EC 3.1.27 | Endoribonucleases Producing 3′-Phosphomonoesters |
| EC 3.1.27.1 | ribonuclease T2 |
| EC 3.1.27.2 | Bacillus subtilis ribonuclease |
| EC 3.1.27.3 | ribonuclease T1 |
| EC 3.1.27.4 | ribonuclease U2 |
| EC 3.1.27.5 | pancreatic ribonuclease |
| EC 3.1.27.6 | Enterobacter ribonuclease |
| EC 3.1.27.7 | ribonuclease F |
| EC 3.1.27.8 | ribonuclease V |
| EC 3.1.27.9 | tRNA-intron endonuclease |
| EC 3.1.27.10 | rRNA endonuclease |
| EC 3.1.30 | Endoribonucleases Active with either Ribo- or |
| | Deoxyribonucleic Acids and Producing 5′- |
| | Phosphomonoesters |
| EC 3.1.30.1 | Aspergillus nuclease S1 |
| EC 3.1.30.2 | Serratia marcescens nuclease |
| EC 3.1.31 | Endoribonucleases Active with either Ribo- or |
| | Deoxyribonucleic Acids and Producing 3′- |
| | Phosphomonoesters |
| EC 3.1.31.1 | micrococcal nuclease |
| EC 3.2 | Glycosylases |
| EC 3.2.1 | Glycosidases, i.e. enzymes hydrolysing O- and |
| | S-glycosyl compounds |
| EC 3.2.1.1 | a-amylase |
| EC 3.2.1.2 | b-amylase |
| EC 3.2.1.3 | glucan 1,4-a-glucosidase |
| EC 3.2.1.4 | cellulase |
| EC 3.2.1.6 | endo-1,3(4)-b-glucanase |
| EC 3.2.1.7 | inulinase |
| EC 3.2.1.8 | endo-1,4-b-xylanase |
| EC 3.2.1.10 | oligo-1,6-glucosidase |
| EC 3.2.1.11 | dextranase |
| EC 3.2.1.14 | chitinase |
| EC 3.2.1.15 | polygalacturonase |
| EC 3.2.1.17 | lysozyme |
| EC 3.2.1.18 | exo-a-sialidase |
| EC 3.2.1.20 | a-glucosidase |
| EC 3.2.1.21 | b-glucosidase |
| EC 3.2.1.22 | a-galactosidase |
| EC 3.2.1.23 | b-galactosidase |
| EC 3.2.1.24 | a-mannosidase |
| EC 3.2.1.25 | b-mannosidase |
| EC 3.2.1.26 | b-fructofuranosidase |
| EC 3.2.1.28 | A,a-trehalase |
| EC 3.2.1.31 | b-glucuronidase |
| EC 3.2.1.32 | xylan endo-1,3-b-xylosidase |
| EC 3.2.1.33 | amylo-1,6-glucosidase |
| EC 3.2.1.35 | hyaluronoglucosaminidase |
| EC 3.2.1.36 | hyaluronoglucuronidase |
| EC 3.2.1.37 | xylan 1,4-b-xylosidase |
| EC 3.2.1.38 | b-D-fucosidase |
| EC 3.2.1.39 | glucan endo-1,3-b-D-glucosidase |
| EC 3.2.1.40 | a-L-rhamnosidase |
| EC 3.2.1.41 | pullulanase |
| EC 3.2.1.42 | GDP-glucosidase |
| EC 3.2.1.43 | b-L-rhamnosidase |
| EC 3.2.1.44 | fucoidanase |
| EC 3.2.1.45 | glucosylceramidase |
| EC 3.2.1.46 | galactosylceramidase |
| EC 3.2.1.47 | galactosylgalactosylglucosylceramidase |
| EC 3.2.1.48 | sucrose a-glucosidase |
| EC 3.2.1.49 | a-N-acetylgalactosaminidase |
| EC 3.2.1.50 | a-N-acetylglucosaminidase |
| EC 3.2.1.51 | a-L-fucosidase |
| EC 3.2.1.52 | b-L-N-acetylhexosaminidase |
| EC 3.2.1.53 | b-N-acetylgalactosaminidase |
| EC 3.2.1.54 | cyclomaltodextrinase |
| EC 3.2.1.55 | a-N-arabinofuranosidase |
| EC 3.2.1.56 | glucuronosyl-disulfoglucosamine glucuronidase |
| EC 3.2.1.57 | isopullulanase |
| EC 3.2.1.58 | glucan 1,3-b-glucosidase |
| EC 3.2.1.59 | glucan endo-1,3-a-glucosidase |
| EC 3.2.1.60 | glucan 1,4-a-maltotetraohydrolase |
| EC 3.2.1.61 | mycodextranase |
| EC 3.2.1.62 | glycosylceramidase |
| EC 3.2.1.63 | 1,2-a-L-fucosidase |
| EC 3.2.1.64 | 2,6-b-fructan 6-levanbiohydrolase |
| EC 3.2.1.65 | levanase |
| EC 3.2.1.66 | quercitrinase |
| EC 3.2.1.67 | galacturan 1,4-a-galacturonidase |
| EC 3.2.1.68 | isoamylase |
| EC 3.2.1.70 | glucan 1,6-a-glucosidase |
| EC 3.2.1.71 | glucan endo-1,2-b-glucosidase |
| EC 3.2.1.72 | xylan 1,3-b-xylosidase |
| EC 3.2.1.73 | licheninase |
| EC 3.2.1.74 | glucan 1,4-b-glucosidase |
| EC 3.2.1.75 | glucan endo-1,6-b-glucosidase |
| EC 3.2.1.76 | L-iduronidase |
| EC 3.2.1.77 | mannan 1,2-(1,3)-a-mannosidase |
| EC 3.2.1.78 | mannan endo-1,4-b-mannosidase |
| EC 3.2.1.80 | fructan b-fructosidase |
| EC 3.2.1.81 | agarase |
| EC 3.2.1.82 | exo-poly-a-galacturonosidase |
| EC 3.2.1.83 | k-carrageenase |
| EC 3.2.1.84 | glucan 1,3-a-glucosidase |
| EC 3.2.1.85 | 6-phospho-b-galactosidase |
| EC 3.2.1.86 | 6-phospho-b-glucosidase |
| EC 3.2.1.87 | capsular-polysaccharide endo-1,3-a-galactosidase |
| EC 3.2.1.88 | b-L-arabinosidase |
| EC 3.2.1.89 | arabinogalactan endo-1,4-b-galactosidase |
| EC 3.2.1.91 | cellulose 1,4-b-cellobiosidase |
| EC 3.2.1.92 | peptidoglycan b-N-acetylmuramidase |
| EC 3.2.1.93 | A,a-phosphotrehalase |
| EC 3.2.1.94 | glucan 1,6-a-isomaltosidase |
| EC 3.2.1.95 | dextran 1,6-a-isomaltotriosidase |
| EC 3.2.1.96 | mannosyl-glycoprotein endo-b-N-acetylglucosaminidase |
| EC 3.2.1.97 | glycopeptide a-N-acetylgalactosaminidase |
| EC 3.2.1.98 | glucan 1,4-a-maltohexaosidase |
| EC 3.2.1.99 | arabinan endo-1,5-a-L-arabinosidase |
| EC 3.2.1.100 | mannan 1,4-mannobiosidase |
| EC 3.2.1.101 | mannan endo-1,6-a-mannosidase |
| EC 3.2.1.102 | blood-group-substance endo-1,4-b-galactosidase |
| EC 3.2.1.103 | keratan-sulfate endo-1,4-b-galactosidase |
| EC 3.2.1.104 | steryl-b-glucosidase |
| EC 3.2.1.105 | strictosidine b-glucosidase |
| EC 3.2.1.106 | mannosyl-oligosaccharide glucosidase |
| EC 3.2.1.107 | protein-glucosylgalactosylhydroxylysine glucosidase |
| EC 3.2.1.108 | lactase |
| EC 3.2.1.109 | endogalactosaminidase |
| EC 3.2.1.110 | mucinaminylserine mucinaminidase |
| EC 3.2.1.111 | 1,3-a-L-fucosidase |
| EC 3.2.1.112 | 2-deoxyglucosidase |
| EC 3.2.1.113 | mannosyl-oligosaccharide 1,2-a-mannosidase |
| EC 3.2.1.114 | mannosyl-oligosaccharide 1,3-1,6-a-mannosidase |
| EC 3.2.1.115 | branched-dextran exo-1,2-a-glucosidase |
| EC 3.2.1.116 | glucan 1,4-a-maltotriohydrolase |
| EC 3.2.1.117 | amygdalin b-glucosidase |
| EC 3.2.1.118 | prunasin b-glucosidase |
| EC 3.2.1.119 | vicianin b-glucosidase |
| EC 3.2.1.120 | oligoxyloglucan b-glycosidase |
| EC 3.2.1.121 | polymannuronate hydrolase |
| EC 3.2.1.122 | maltose-6′-phosphate glucosidase |
| EC 3.2.1.123 | endoglycosylceramidase |
| EC 3.2.1.124 | 3-deoxy-2-octulosonidase |
| EC 3.2.1.125 | raucaffricine b-glucosidase |
| EC 3.2.1.126 | coniferin b-glucosidase |
| EC 3.2.1.127 | 1,6-a-L-fucosidase |
| EC 3.2.1.128 | glycyrrhizinate b-glucuronidase |
| EC 3.2.1.129 | endo-a-sialidase |
| EC 3.2.1.130 | glycoprotein endo-a-1,2-mannosidase |
| EC 3.2.1.131 | xylan a-1,2-glucuronosidase |
| EC 3.2.1.132 | chitosanase |
| EC 3.2.1.133 | glucan 1,4-a-maltohydrolase |
| EC 3.2.1.134 | difructose-anhydride synthase |
| EC 3.2.1.135 | neopullulanase |
| EC 3.2.1.136 | glucuronoarabinoxylan endo-1,4-b-xylanase |
| EC 3.2.1.137 | mannan exo-1,2-1,6-a-mannosidase |
| EC 3.2.1.139 | a-glucuronidase |
| EC 3.2.1.140 | lacto-N-biosidase |
| EC 3.2.1.141 | 4-a-D-{(14)-a-D-glucano}trehalose trehalohydrolase |
| EC 3.2.1.142 | limit dextrinase |
| EC 3.2.1.143 | poly(ADP-ribose) glycohydrolase |
| EC 3.2.1.144 | 3-deoxyoctulosonase |
| EC 3.2.1.145 | galactan 1,3-b-galactosidase |
| EC 3.2.1.146 | b-galactofuranosidase |
| EC 3.2.1.147 | thioglucosidase |
| EC 3.2.1.149 | b-primeverosidase |
| EC 3.2.1.150 | oligoxyloglucan reducing-end-specific cellobiohydrolase |
| EC 3.2.1.151 | xyloglucan-specific endo-b-1,4-glucanase |
| EC 3.2.2 | Hydrolysing N-Glycosyl Compounds |
| EC 3.2.2.1 | purine nucleosidase |
| EC 3.2.2.2 | inosine nucleosidase |
| EC 3.2.2.3 | uridine nucleosidase |
| EC 3.2.2.4 | AMP nucleosidase |
| EC 3.2.2.5 | NAD+ nucleosidase |
| EC 3.2.2.6 | NAD(P)+ nucleosidase |
| EC 3.2.2.7 | adenosine nucleosidase |
| EC 3.2.2.8 | ribosylpyrimidine nucleosidase |
| EC 3.2.2.9 | adenosylhomocysteine nucleosidase |
| EC 3.2.2.10 | pyrimidine-5′-nucleotide nucleosidase |
| EC 3.2.2.11 | b-aspartyl-N-acetylglucosaminidase |
| EC 3.2.2.12 | inosinate nucleosidase |
| EC 3.2.2.13 | 1-methyladenosine nucleosidase |
| EC 3.2.2.14 | NMN nucleosidase |
| EC 3.2.2.15 | DNA-deoxyinosine glycosylase |
| EC 3.2.2.16 | methylthioadenosine nucleosidase |
| EC 3.2.2.17 | deoxyribodipyrimidine endonucleosidase |
| EC 3.2.2.19 | ADP-ribosylarginine hydrolase |
| EC 3.2.2.20 | DNA-3-methyladenine glycosylase I |
| EC 3.2.2.21 | DNA-3-methyladenine glycosylase II |
| EC 3.2.2.22 | rRNA N-glycosylase |
| EC 3.2.2.23 | DNA-formamidopyrimidine glycosylase |
| EC 3.2.2.24 | ADP-ribosyl-[dinitrogen reductase] hydrolase |
| EC 3.2.3 | Hydrolysing S-Glycosyl Compounds (discontinued) |
| EC 3.3 | Acting on Ether Bonds |
| EC 3.3.1 | Thioether and Trialkylsulfonium Hydrolases |
| EC 3.3.1.1 | adenosylhomocysteinase |
| EC 3.3.1.2 | adenosylmethionine hydrolase |
| EC 3.3.2 | Ether Hydrolases |
| EC 3.3.2.1 | isochorismatase |
| EC 3.3.2.2 | alkenylglycerophosphocholine hydrolase |
| EC 3.3.2.3 | epoxide hydrolase |
| EC 3.3.2.4 | trans-epoxysuccinate hydrolase |
| EC 3.3.2.5 | alkenylglycerophosphoethanolamine hydrolase |
| EC 3.3.2.6 | leukotriene-A4 hydrolase |
| EC 3.3.2.7 | hepoxilin-epoxide hydrolase |
| EC 3.3.2.8 | limonene-1,2-epoxide hydrolase |
| EC 3.4 | Acting on peptide bonds (Peptidases) |
| EC 3.4.11 | Aminopeptidases |
| EC 3.4.11.1 | Leucyl aminopeptidase |
| EC 3.4.11.2 | Membrane alanyl aminopeptidase |
| EC 3.4.11.3 | Cystinyl aminopeptidase |
| EC 3.4.11.4 | Tripeptide aminopeptidase |
| EC 3.4.11.5 | Prolyl aminopeptidase |
| EC 3.4.11.6 | Arginyl aminopeptidase |
| EC 3.4.11.7 | Glutamyl aminopeptidase |
| EC 3.4.11.9 | Xaa-Pro aminopeptidase |
| EC 3.4.11.10 | Bacterial leucyl aminopeptidase |
| EC 3.4.11.13 | Clostridial aminopeptidase |
| EC 3.4.11.14 | Cytosol alanyl aminopeptidase |
| EC 3.4.11.15 | Lysyl aminopeptidase |
| EC 3.4.11.16 | Xaa-Trp aminopeptidase |
| EC 3.4.11.17 | Tryptophanyl aminopeptidase |
| EC 3.4.11.18 | Methionyl aminopeptidase |
| EC 3.4.11.19 | D-Stereospecific aminopeptidase |
| EC 3.4.11.20 | Aminopeptidase Ey |
| EC 3.4.11.21 | aspartyl aminopeptidase |
| EC 3.4.11.22 | Aminopeptidase I |
| EC 3.4.11.23 | PepB aminopeptidase |
| EC 3.4.13 | Dipeptidases |
| EC 3.4.13.3 | Xaa-His dipeptidase |
| EC 3.4.13.4 | Xaa-Arg dipeptidase |
| EC 3.4.13.5 | Xaa-Methyl-His dipeptidase |
| EC 3.4.13.7 | Glu-Glu dipeptidase |
| EC 3.4.13.9 | Xaa-Pro dipeptidase |
| EC 3.4.13.12 | Met-Xaa dipeptidase |
| EC 3.4.13.17 | non-stereospecific dipeptidase |
| EC 3.4.13.18 | cytosol nonspecific dipeptidase |
| EC 3.4.13.19 | membrane dipeptidase |
| EC 3.4.13.20 | b-Ala-His dipeptidase |
| EC 3.4.13.21 | dipeptidase E |
| EC 3.4.14 | Dipeptidyl-peptidases and tripeptidyl-peptidases |
| EC 3.4.14.1 | Dipeptidyl-peptidase I |
| EC 3.4.14.2 | Dipeptidyl-peptidase II |
| EC 3.4.14.4 | Dipeptidyl-peptidase III |
| EC 3.4.14.5 | Dipeptidyl-peptidase IV |
| EC 3.4.14.6 | Dipeptidyl-dipeptidase |
| EC 3.4.14.9 | Tripeptidyl-peptidase I |
| EC 3.4.14.10 | Tripeptidyl-peptidase II |
| EC 3.4.14.11 | Xaa-Pro dipeptidyl-peptidase |
| EC 3.4.15 | Peptidyl-dipeptidases |
| EC 3.4.15.1 | Peptidyl-dipeptidase A |
| EC 3.4.15.4 | Peptidyl-dipeptidase B |
| EC 3.4.15.5 | Peptidyl-dipeptidase Dcp |
| EC 3.4.16 | Serine-type carboxypeptidases |
| EC 3.4.16.2 | Lysosomal Pro-Xaa carboxypeptidase |
| EC 3.4.16.4 | Serine-type D-Ala-D-Ala carboxypeptidase |
| EC 3.4.16.5 | Carboxypeptidase C |
| EC 3.4.16.6 | Carboxypeptidase D |
| EC 3.4.17 | Metallocarboxypeptidases |
| EC 3.4.17.1 | Carboxypeptidase A |
| EC 3.4.17.2 | Carboxypeptidase B |
| EC 3.4.17.3 | Lysine carboxypeptidase |
| EC 3.4.17.4 | Gly-Xaa carboxypeptidase |
| EC 3.4.17.6 | Alanine carboxypeptidase |
| EC 3.4.17.8 | Muramoylpentapeptide carboxypeptidase |
| EC 3.4.17.10 | Carboxypeptidase E |
| EC 3.4.17.11 | Glutamate carboxypeptidase |
| EC 3.4.17.12 | Carboxypeptidase M |
| EC 3.4.17.13 | Muramoyltetrapeptide carboxypeptidase |
| EC 3.4.17.14 | Zinc D-Ala-D-Ala carboxypeptidase |
| EC 3.4.17.15 | Carboxypeptidase A2 |
| EC 3.4.17.16 | Membrane Pro-Xaa carboxypeptidase |
| EC 3.4.17.17 | Tubulinyl-Tyr carboxypeptidase |
| EC 3.4.17.18 | Carboxypeptidase T |
| EC 3.4.17.19 | Carboxypeptidase Taq |
| EC 3.4.17.20 | Carboxypeptidase U |
| EC 3.4.17.21 | glutamate carboxypeptidase II |
| EC 3.4.17.22 | Metallocarboxypeptidase D |
| EC 3.4.18 | Cysteine-type carboxypeptidases |
| EC 3.4.18.1 | Cathepsin X |
| EC 3.4.19 | Omega peptidases |
| EC 3.4.19.1 | Acylaminoacyl-peptidase |
| EC 3.4.19.2 | Peptidyl-glycinamidase |
| EC 3.4.19.3 | Pyroglutamyl-peptidase I |
| EC 3.4.19.5 | b-Aspartyl-peptidase |
| EC 3.4.19.6 | Pyroglutamyl-peptidase II |
| EC 3.4.19.7 | N-Formylmethionyl-peptidase |
| EC 3.4.19.9 | g-Glutamyl hydrolase |
| EC 3.4.19.11 | g-D-Glutamyl-meso-diaminopimelate peptidase I |
| EC 3.4.19.12 | ubiquitinyl hydrolase 1 |
| EC 3.4.21 | Serine endopeptidases |
| EC 3.4.21.1 | Chymotrypsin |
| EC 3.4.21.2 | Chymotrypsin C |
| EC 3.4.21.3 | Metridin |
| EC 3.4.21.4 | Trypsin |
| EC 3.4.21.5 | Thrombin |
| EC 3.4.21.6 | Coagulation Factor Xa |
| EC 3.4.21.7 | Plasmin |
| EC 3.4.21.9 | Enteropeptidase |
| EC 3.4.21.10 | Acrosin |
| EC 3.4.21.12 | a-Lytic endopeptidase |
| EC 3.4.21.19 | Glutamyl endopeptidase |
| EC 3.4.21.20 | Cathepsin G |
| EC 3.4.21.21 | Coagulation Factor VIIa |
| EC 3.4.21.22 | Coagulation Factor IXa |
| EC 3.4.21.25 | Cucumisin |
| EC 3.4.21.26 | Prolyl oligopeptidase |
| EC 3.4.21.27 | Coagulation Factor XIa |
| EC 3.4.21.32 | Brachyurin |
| EC 3.4.21.34 | Plasma kallikrein |
| EC 3.4.21.35 | Tissue kallikrein |
| EC 3.4.21.36 | Pancreatic elastase |
| EC 3.4.21.37 | Leukocyte elastase |
| EC 3.4.21.38 | Coagulation Factor XIIa |
| EC 3.4.21.39 | Chymase |
| EC 3.4.21.41 | Complement subcomponent C |
| EC 3.4.21.42 | Complement subcomponent C |
| EC 3.4.21.43 | Classical-complement-pathway C3/C5 convertase |
| EC 3.4.21.45 | Complement Factor I |
| EC 3.4.21.46 | Complement Factor D |
| EC 3.4.21.47 | Alternative-complement-pathway C3/C5 convertase |
| EC 3.4.21.48 | Cerevisin |
| EC 3.4.21.49 | Hypodermin C |
| EC 3.4.21.50 | Lysyl endopeptidase |
| EC 3.4.21.53 | Endopeptidase La |
| EC 3.4.21.54 | g-Renin |
| EC 3.4.21.55 | Venombin AB |
| EC 3.4.21.57 | Leucyl endopeptidase |
| EC 3.4.21.59 | Tryptase |
| EC 3.4.21.60 | Scutelarin |
| EC 3.4.21.61 | Kexin |
| EC 3.4.21.62 | Subtilisin |
| EC 3.4.21.63 | Oryzin |
| EC 3.4.21.64 | Endopeptidase K |
| EC 3.4.21.65 | Thermomycolin |
| EC 3.4.21.66 | Thermitase |
| EC 3.4.21.67 | Endopeptidase So |
| EC 3.4.21.68 | t-Plasminogen activator |
| EC 3.4.21.69 | Protein C (activated) |
| EC 3.4.21.70 | Pancreatic endopeptidase E |
| EC 3.4.21.71 | Pancreatic elastase II |
| EC 3.4.21.72 | IgA-specific serine endopeptidase |
| EC 3.4.21.73 | u-Plasminogen activator |
| EC 3.4.21.74 | Venombin A |
| EC 3.4.21.75 | Furin |
| EC 3.4.21.76 | Myeloblastin |
| EC 3.4.21.77 | Semenogelase |
| EC 3.4.21.78 | Granzyme A |
| EC 3.4.21.79 | Granzyme B |
| EC 3.4.21.80 | Streptogrisin A |
| EC 3.4.21.81 | Streptogrisin B |
| EC 3.4.21.82 | Glutamyl endopeptidase II |
| EC 3.4.21.83 | Oligopeptidase B |
| EC 3.4.21.84 | Limulus clotting factor |
| EC 3.4.21.85 | Limulus clotting factor |
| EC 3.4.21.86 | Limulus clotting enzyme |
| EC 3.4.21.87 | Omptin |
| EC 3.4.21.88 | Repressor LexA |
| EC 3.4.21.89 | Signal peptidase I |
| EC 3.4.21.90 | Togavirin |
| EC 3.4.21.91 | Flavivirin |
| EC 3.4.21.92 | Endopeptidase Clp |
| EC 3.4.21.93 | Proprotein convertase 1 |
| EC 3.4.21.94 | Proprotein convertase 2 |
| EC 3.4.21.95 | Snake venom factor V activator |
| EC 3.4.21.96 | Lactocepin |
| EC 3.4.21.97 | assemblin |
| EC 3.4.21.98 | hepacivirin |
| EC 3.4.21.99 | spermosin |
| EC 3.4.21.100 | pseudomonalisin |
| EC 3.4.21.101 | xanthomonalisin |
| EC 3.4.21.102 | C-terminal processing peptidase |
| EC 3.4.21.103 | physarolisin |
| EC 3.4.22 | Cysteine endopeptidases |
| EC 3.4.22.1 | Cathepsin B |
| EC 3.4.22.2 | papain |
| EC 3.4.22.3 | Ficain |
| EC 3.4.22.6 | Chymopapain |
| EC 3.4.22.7 | Asclepain |
| EC 3.4.22.8 | Clostripain |
| EC 3.4.22.10 | Streptopain |
| EC 3.4.22.14 | Actinidain |
| EC 3.4.22.15 | Cathepsin L |
| EC 3.4.22.16 | Cathepsin H |
| EC 3.4.22.24 | Cathepsin T |
| EC 3.4.22.25 | Glycyl endopeptidase |
| EC 3.4.22.26 | Cancer procoagulant |
| EC 3.4.22.27 | Cathepsin S |
| EC 3.4.22.28 | Picornain 3C |
| EC 3.4.22.29 | Picornain 2A |
| EC 3.4.22.30 | Caricain |
| EC 3.4.22.31 | Ananain |
| EC 3.4.22.32 | Stem bromelain |
| EC 3.4.22.33 | Fruit bromelain |
| EC 3.4.22.34 | legumain |
| EC 3.4.22.35 | Histolysain |
| EC 3.4.22.36 | Caspase-1 |
| EC 3.4.22.37 | Gingipain R |
| EC 3.4.22.38 | Cathepsin K |
| EC 3.4.22.39 | adenain |
| EC 3.4.22.40 | bleomycin hydrolase |
| EC 3.4.22.41 | cathepsin F |
| EC 3.4.22.42 | cathepsin O |
| EC 3.4.22.43 | cathepsin V |
| EC 3.4.22.44 | nuclear-inclusion-a endopeptidase |
| EC 3.4.22.45 | helper-component proteinase |
| EC 3.4.22.46 | L-peptidase |
| EC 3.4.22.47 | gingipain K |
| EC 3.4.22.48 | staphopain |
| EC 3.4.22.49 | separase |
| EC 3.4.22.50 | V-cath endopeptidase |
| EC 3.4.22.51 | cruzipain |
| EC 3.4.22.52 | calpain-1 |
| EC 3.4.22.53 | calpain-2 |
| EC 3.4.23 | Aspartic endopeptidases |
| EC 3.4.23.1 | Pepsin A |
| EC 3.4.23.2 | Pepsin B |
| EC 3.4.23.3 | Gastricsin |
| EC 3.4.23.4 | Chymosin |
| EC 3.4.23.5 | Cathepsin D |
| EC 3.4.23.12 | Nepenthesin |
| EC 3.4.23.15 | Renin |
| EC 3.4.23.16 | HIV-1 retropepsin |
| EC 3.4.23.17 | Pro-opiomelanocortin converting enzyme |
| EC 3.4.23.18 | Aspergillopepsin I |
| EC 3.4.23.19 | Aspergillopepsin II |
| EC 3.4.23.20 | Penicillopepsin |
| EC 3.4.23.21 | Rhizopuspepsin |
| EC 3.4.23.22 | Endothiapepsin |
| EC 3.4.23.23 | Mucorpepsin |
| EC 3.4.23.24 | Candidapepsin |
| EC 3.4.23.25 | Saccharopepsin |
| EC 3.4.23.26 | Rhodotorulapepsin |
| EC 3.4.23.28 | Acrocylindropepsin |
| EC 3.4.23.29 | Polyporopepsin |
| EC 3.4.23.30 | Pycnoporopepsin |
| EC 3.4.23.31 | Scytalidopepsin A |
| EC 3.4.23.32 | Scytalidopepsin B |
| EC 3.4.23.34 | Cathepsin E |
| EC 3.4.23.35 | Barrierpepsin |
| EC 3.4.23.36 | Signal peptidase II |
| EC 3.4.23.38 | Plasmepsin I |
| EC 3.4.23.39 | Plasmepsin II |
| EC 3.4.23.40 | Phytepsin |
| EC 3.4.23.41 | yapsin 1 |
| EC 3.4.23.42 | thermopsin |
| EC 3.4.23.43 | prepilin peptidase |
| EC 3.4.23.44 | nodavirus endopeptidase |
| EC 3.4.23.45 | memapsin 1 |
| EC 3.4.23.46 | memapsin 2 |
| EC 3.4.23.47 | HIV-2 retropepsin |
| EC 3.4.23.48 | plasminogen activator Pla |
| EC 3.4.24 | Metalloendopeptidases |
| EC 3.4.24.1 | Atrolysin A |
| EC 3.4.24.3 | Microbial collagenase |
| EC 3.4.24.6 | Leucolysin |
| EC 3.4.24.7 | Interstitial collagenase |
| EC 3.4.24.11 | Neprilysin |
| EC 3.4.24.12 | Envelysin |
| EC 3.4.24.13 | IgA-specific metalloendopeptidase |
| EC 3.4.24.14 | Procollagen N-endopeptidase |
| EC 3.4.24.15 | Thimet oligopeptidase |
| EC 3.4.24.16 | Neurolysin |
| EC 3.4.24.17 | Stromelysin 1 |
| EC 3.4.24.18 | Meprin A |
| EC 3.4.24.19 | Procollagen C-endopeptidase |
| EC 3.4.24.20 | Peptidyl-Lys metalloendopeptidase |
| EC 3.4.24.21 | Astacin |
| EC 3.4.24.22 | Stromelysin 2 |
| EC 3.4.24.23 | Matrilysin |
| EC 3.4.24.24 | Gelatinase A |
| EC 3.4.24.25 | Vibriolysin |
| EC 3.4.24.26 | Pseudolysin |
| EC 3.4.24.27 | Thermolysin |
| EC 3.4.24.28 | Bacillolysin |
| EC 3.4.24.29 | Aureolysin |
| EC 3.4.24.30 | Coccolysin |
| EC 3.4.24.31 | Mycolysin |
| EC 3.4.24.32 | b-Lytic metalloendopeptidase |
| EC 3.4.24.33 | Peptidyl-Asp metalloendopeptidase |
| EC 3.4.24.34 | Neutrophil collagenase |
| EC 3.4.24.35 | Gelatinase B |
| EC 3.4.24.36 | Leishmanolysin |
| EC 3.4.24.37 | Saccharolysin |
| EC 3.4.24.38 | gametolysin |
| EC 3.4.24.39 | Deuterolysin |
| EC 3.4.24.40 | Serralysin |
| EC 3.4.24.41 | Atrolysin B |
| EC 3.4.24.42 | Atrolysin C |
| EC 3.4.24.43 | Atroxase |
| EC 3.4.24.44 | Atrolysin E |
| EC 3.4.24.45 | Atrolysin F |
| EC 3.4.24.46 | Adamalysin |
| EC 3.4.24.47 | Horrilysin |
| EC 3.4.24.48 | Ruberlysin |
| EC 3.4.24.49 | Bothropasin |
| EC 3.4.24.50 | Bothrolysin |
| EC 3.4.24.51 | Ophiolysin |
| EC 3.4.24.52 | Trimerelysin I |
| EC 3.4.24.53 | Trimerelysin II |
| EC 3.4.24.54 | Mucrolysin |
| EC 3.4.24.55 | Pitrilysin |
| EC 3.4.24.56 | Insulysin |
| EC 3.4.24.57 | O-Sialoglycoprotein endopeptidase |
| EC 3.4.24.58 | Russellysin |
| EC 3.4.24.59 | Mitochondrial intermediate peptidase |
| EC 3.4.24.60 | Dactylysin |
| EC 3.4.24.61 | Nardilysin |
| EC 3.4.24.62 | Magnolysin |
| EC 3.4.24.63 | Meprin B |
| EC 3.4.24.64 | Mitochondrial processing peptidase |
| EC 3.4.24.65 | Macrophage elastase |
| EC 3.4.24.66 | Choriolysin L |
| EC 3.4.24.67 | Choriolysin H |
| EC 3.4.24.68 | Tentoxilysin |
| EC 3.4.24.69 | Bontoxilysin |
| EC 3.4.24.70 | Oligopeptidase A |
| EC 3.4.24.71 | Endothelin-converting enzyme |
| EC 3.4.24.72 | Fibrolase |
| EC 3.4.24.73 | Jararhagin |
| EC 3.4.24.74 | Fragilysin |
| EC 3.4.24.75 | Lysostaphin |
| EC 3.4.24.76 | flavastacin |
| EC 3.4.24.77 | snapalysin |
| EC 3.4.24.78 | gpr endopeptidase |
| EC 3.4.24.79 | pappalysin-1 |
| EC 3.4.24.80 | membrane-type matrix metalloproteinase-1 |
| EC 3.4.24.81 | ADAM10 endopeptidase |
| EC 3.4.24.82 | ADAMTS-4 endopeptidase |
| EC 3.4.24.83 | anthrax lethal factor endopeptidase |
| EC 3.4.24.84 | Ste24 endopeptidase |
| EC 3.4.24.85 | S2P endopeptidase |
| EC 3.4.24.86 | ADAM 17 endopeptidase |
| EC 3.4.25 | Threonine endopeptidases |
| EC 3.4.25.1 | proteasome endopeptidase complex |
| EC 3.5 | Acting on Carbon-Nitrogen Bonds, other than Peptide |
| | Bonds |
| EC 3.5.1 | In Linear Amides |
| EC 3.5.1.1 | asparaginase |
| EC 3.5.1.2 | glutaminase |
| EC 3.5.1.3 | w-amidase |
| EC 3.5.1.4 | amidase |
| EC 3.5.1.5 | urease |
| EC 3.5.1.6 | b-ureidopropionase |
| EC 3.5.1.7 | ureidosuccinase |
| EC 3.5.1.8 | formylaspartate deformylase |
| EC 3.5.1.9 | arylformamidase |
| EC 3.5.1.10 | formyltetrahydrofolate deformylase |
| EC 3.5.1.11 | penicillin amidase |
| EC 3.5.1.12 | biotinidase |
| EC 3.5.1.13 | aryl-acylamidase |
| EC 3.5.1.14 | aminoacylase |
| EC 3.5.1.15 | aspartoacylase |
| EC 3.5.1.16 | acetylornithine deacetylase |
| EC 3.5.1.17 | acyl-lysine deacylase |
| EC 3.5.1.18 | succinyl-diaminopimelate desuccinylase |
| EC 3.5.1.19 | nicotinamidase |
| EC 3.5.1.20 | citrullinase |
| EC 3.5.1.21 | N-acetyl-b-alanine deacetylase |
| EC 3.5.1.22 | pantothenase |
| EC 3.5.1.23 | ceramidase |
| EC 3.5.1.24 | choloylglycine hydrolase |
| EC 3.5.1.25 | N-acetylglucosamine-6-phosphate deacetylase |
| EC 3.5.1.26 | N4-(b-N-acetylglucosaminyl)-L-asparaginase |
| EC 3.5.1.27 | N-formylmethionylaminoacyl-tRNA deformylase |
| EC 3.5.1.28 | N-acetylmuramoyl-L-alanine amidase |
| EC 3.5.1.29 | 2-(acetamidomethylene)succinate hydrolase |
| EC 3.5.1.30 | 5-aminopentanamidase |
| EC 3.5.1.31 | formylmethionine deformylase |
| EC 3.5.1.32 | hippurate hydrolase |
| EC 3.5.1.33 | N-acetylglucosamine deacetylase |
| EC 3.5.1.35 | D-glutaminase |
| EC 3.5.1.36 | N-methyl-2-oxoglutaramate hydrolase |
| EC 3.5.1.38 | glutamin-(asparagin-)ase |
| EC 3.5.1.39 | alkylamidase |
| EC 3.5.1.40 | acylagmatine amidase |
| EC 3.5.1.41 | chitin deacetylase |
| EC 3.5.1.42 | nicotinamide-nucleotide amidase |
| EC 3.5.1.43 | peptidyl-glutaminase |
| EC 3.5.1.44 | protein-glutamine glutaminase |
| EC 3.5.1.46 | 6-aminohexanoate-dimer hydrolase |
| EC 3.5.1.47 | N-acetyldiaminopimelate deacetylase |
| EC 3.5.1.48 | acetylspermidine deacetylase |
| EC 3.5.1.49 | formamidase |
| EC 3.5.1.50 | pentanamidase |
| EC 3.5.1.51 | 4-acetamidobutyryl-CoA deacetylase |
| EC 3.5.1.52 | peptide-N4-(N-acetyl-b-glucosaminyl)asparagine amidase |
| EC 3.5.1.53 | N-carbamoylputrescine amidase |
| EC 3.5.1.54 | allophanate hydrolase |
| EC 3.5.1.55 | long-chain-fatty-acyl-glutamate deacylase |
| EC 3.5.1.56 | N,N-dimethylformamidase |
| EC 3.5.1.57 | tryptophanamidase |
| EC 3.5.1.58 | N-benzyloxycarbonylglycine hydrolase |
| EC 3.5.1.59 | N-carbamoylsarcosine amidase |
| EC 3.5.1.60 | N-(long-chain-acyl)ethanolamine deacylase |
| EC 3.5.1.61 | mimosinase |
| EC 3.5.1.62 | acetylputrescine deacetylase |
| EC 3.5.1.63 | 4-acetamidobutyrate deacetylase |
| EC 3.5.1.64 | Na-benzyloxycarbonylleucine hydrolase |
| EC 3.5.1.65 | theanine hydrolase |
| EC 3.5.1.66 | 2-(hydroxymethyl)-3-(acetamidomethylene)succinate |
| | hydrolase |
| EC 3.5.1.67 | 4-methyleneglutaminase |
| EC 3.5.1.68 | N-formylglutamate deformylase |
| EC 3.5.1.69 | glycosphingolipid deacylase |
| EC 3.5.1.70 | aculeacin-A deacylase |
| EC 3.5.1.71 | N-feruloylglycine deacylase |
| EC 3.5.1.72 | D-benzoylarginine-4-nitroanilide amidase |
| EC 3.5.1.73 | carnitinamidase |
| EC 3.5.1.74 | chenodeoxycholoyltaurine hydrolase |
| EC 3.5.1.75 | urethanase |
| EC 3.5.1.76 | arylalkyl acylamidase |
| EC 3.5.1.77 | N-carbamoyl-D-amino acid hydrolase |
| EC 3.5.1.78 | glutathionylspermidine amidase |
| EC 3.5.1.79 | phthalyl amidase |
| EC 3.5.1.81 | N-acyl-D-amino-acid deacylase |
| EC 3.5.1.82 | N-acyl-D-glutamate deacylase |
| EC 3.5.1.83 | N-acyl-D-aspartate deacylase |
| EC 3.5.1.84 | biuret amidohydrolase |
| EC 3.5.1.85 | (S)—N-acetyl-1-phenylethylamine hydrolase |
| EC 3.5.1.86 | mandelamide amidase |
| EC 3.5.1.87 | N-carbamoyl-L-amino-acid hydrolase |
| EC 3.5.1.88 | peptide deformylase |
| EC 3.5.1.89 | N-acetylglucosaminylphosphatidylinositol deacetylase |
| EC 3.5.1.90 | adenosylcobinamide hydrolase |
| EC 3.5.2 | In Cyclic Amides |
| EC 3.5.2.1 | barbiturase |
| EC 3.5.2.2 | dihydropyrimidinase |
| EC 3.5.2.3 | dihydroorotase |
| EC 3.5.2.4 | carboxymethylhydantoinase |
| EC 3.5.2.5 | allantoinase |
| EC 3.5.2.6 | b-lactamase |
| EC 3.5.2.7 | imidazolonepropionase |
| EC 3.5.2.9 | 5-oxoprolinase (ATP-hydrolysing) |
| EC 3.5.2.10 | creatininase |
| EC 3.5.2.11 | L-lysine-lactamase |
| EC 3.5.2.12 | 6-aminohexanoate-cyclic-dimer hydrolase |
| EC 3.5.2.13 | 2,5-dioxopiperazine hydrolase |
| EC 3.5.2.14 | N-methylhydantoinase (ATP-hydrolysing) |
| EC 3.5.2.15 | cyanuric acid amidohydrolase |
| EC 3.5.2.16 | maleimide hydrolase |
| EC 3.5.2.17 | hydroxyisourate hydrolase |
| EC 3.5.3 | In Linear Amidines |
| EC 3.5.3.1 | arginase |
| EC 3.5.3.2 | guanidinoacetase |
| EC 3.5.3.3 | creatinase |
| EC 3.5.3.4 | allantoicase |
| EC 3.5.3.5 | formiminoaspartate deiminase |
| EC 3.5.3.6 | arginine deiminase |
| EC 3.5.3.7 | guanidinobutyrase |
| EC 3.5.3.8 | formimidoylglutamase |
| EC 3.5.3.9 | allantoate deiminase |
| EC 3.5.3.10 | D-arginase |
| EC 3.5.3.11 | agmatinase |
| EC 3.5.3.12 | agmatine deiminase |
| EC 3.5.3.13 | formiminoglutamate deiminase |
| EC 3.5.3.14 | amidinoaspartase |
| EC 3.5.3.15 | protein-arginine deiminase |
| EC 3.5.3.16 | methylguanidinase |
| EC 3.5.3.17 | guanidinopropionase |
| EC 3.5.3.18 | dimethylargininase |
| EC 3.5.3.19 | ureidoglycolate hydrolase |
| EC 3.5.3.20 | diguanidinobutanase |
| EC 3.5.3.21 | methylenediurea deaminase |
| EC 3.5.3.22 | proclavaminate amidinohydrolase |
| EC 3.5.4 | In Cyclic Amidines |
| EC 3.5.4.1 | cytosine deaminase |
| EC 3.5.4.2 | adenine deaminase |
| EC 3.5.4.3 | guanine deaminase |
| EC 3.5.4.4 | adenosine deaminase |
| EC 3.5.4.5 | cytidine deaminase |
| EC 3.5.4.6 | AMP deaminase |
| EC 3.5.4.7 | ADP deaminase |
| EC 3.5.4.8 | aminoimidazolase |
| EC 3.5.4.9 | methenyltetrahydrofolate cyclohydrolase |
| EC 3.5.4.10 | IMP cyclohydrolase |
| EC 3.5.4.11 | pterin deaminase |
| EC 3.5.4.12 | dCMP deaminase |
| EC 3.5.4.13 | dCTP deaminase |
| EC 3.5.4.14 | deoxycytidine deaminase |
| EC 3.5.4.15 | guanosine deaminase |
| EC 3.5.4.16 | GTP cyclohydrolase I |
| EC 3.5.4.17 | adenosine-phosphate deaminase |
| EC 3.5.4.18 | ATP deaminase |
| EC 3.5.4.19 | phosphoribosyl-AMP cyclohydrolase |
| EC 3.5.4.20 | pyrithiamine deaminase |
| EC 3.5.4.21 | creatinine deaminase |
| EC 3.5.4.22 | 1-pyrroline-4-hydroxy-2-carboxylate deaminase |
| EC 3.5.4.23 | blasticidin-S deaminase |
| EC 3.5.4.24 | sepiapterin deaminase |
| EC 3.5.4.25 | GTP cyclohydrolase II |
| EC 3.5.4.26 | diaminohydroxyphosphoribosylaminopyrimidine |
| | deaminase |
| EC 3.5.4.27 | methenyltetrahydromethanopterin cyclohydrolase |
| EC 3.5.4.28 | S-adenosylhomocysteine deaminase |
| EC 3.5.4.29 | GTP cyclohydrolase IIa |
| EC 3.5.4.30 | dCTP deaminase (dUMP-forming) |
| EC 3.5.5 | In Nitriles |
| EC 3.5.5.1 | nitrilase |
| EC 3.5.5.2 | ricinine nitrilase |
| EC 3.5.5.4 | cyanoalanine nitrilase |
| EC 3.5.5.5 | arylacetonitrilase |
| EC 3.5.5.6 | bromoxynil nitrilase |
| EC 3.5.5.7 | aliphatic nitrilase |
| EC 3.5.5.8 | thiocyanate hydrolase |
| EC 3.5.99 | In Other Compounds |
| EC 3.5.99.1 | riboflavinase |
| EC 3.5.99.2 | thiaminase |
| EC 3.5.99.3 | hydroxydechloroatrazine ethylaminohydrolase |
| EC 3.5.99.4 | N-isopropylammelide isopropylaminohydrolase |
| EC 3.5.99.5 | 2-aminomuconate deaminase |
| EC 3.5.99.6 | glucosamine-6-phosphate deaminase |
| EC 3.5.99.7 | 1-aminocyclopropane-1-carboxylate deaminase |
| EC 3.6 | Acting on Acid Anhydrides |
| EC 3.6.1 | In Phosphorus-Containing Anhydrides |
| EC 3.6.1.1 | inorganic diphosphatase |
| EC 3.6.1.2 | trimetaphosphatase |
| EC 3.6.1.3 | adenosinetriphosphatase |
| EC 3.6.1.5 | apyrase |
| EC 3.6.1.6 | nucleoside-diphosphatase |
| EC 3.6.1.7 | acylphosphatase |
| EC 3.6.1.8 | ATP diphosphatase |
| EC 3.6.1.9 | nucleotide diphosphatase |
| EC 3.6.1.10 | endopolyphosphatase |
| EC 3.6.1.11 | exopolyphosphatase |
| EC 3.6.1.12 | dCTP diphosphatase |
| EC 3.6.1.13 | ADP-ribose diphosphatase |
| EC 3.6.1.14 | adenosine-tetraphosphatase |
| EC 3.6.1.15 | nucleoside-triphosphatase |
| EC 3.6.1.16 | CDP-glycerol diphosphatase |
| EC 3.6.1.17 | bis(5′-nucleosyl)-tetraphosphatase (asymmetrical) |
| EC 3.6.1.18 | FAD diphosphatase |
| EC 3.6.1.19 | nucleoside-triphosphate diphosphatase |
| EC 3.6.1.20 | 5′-acylphosphoadenosine hydrolase |
| EC 3.6.1.21 | ADP-sugar diphosphatase |
| EC 3.6.1.22 | NAD+ diphosphatase |
| EC 3.6.1.23 | dUTP diphosphatase |
| EC 3.6.1.24 | nucleoside phosphoacylhydrolase |
| EC 3.6.1.25 | triphosphatase |
| EC 3.6.1.26 | CDP-diacylglycerol diphosphatase |
| EC 3.6.1.27 | undecaprenyl-diphosphatase |
| EC 3.6.1.28 | thiamine-triphosphatase |
| EC 3.6.1.29 | bis(5′-adenosyl)-triphosphatase |
| EC 3.6.1.30 | m7G(5′)pppN diphosphatase |
| EC 3.6.1.31 | phosphoribosyl-ATP diphosphatase |
| EC 3.6.1.39 | thymidine-triphosphatase |
| EC 3.6.1.40 | guanosine-5′-triphosphate,3′-diphosphate diphosphatase |
| EC 3.6.1.41 | bis(5′-nucleosyl)-tetraphosphatase (symmetrical) |
| EC 3.6.1.42 | guanosine-diphosphatase |
| EC 3.6.1.43 | dolichyldiphosphatase |
| EC 3.6.1.44 | oligosaccharide-diphosphodolichol diphosphatase |
| EC 3.6.1.45 | UDP-sugar diphosphatase |
| EC 3.6.1.52 | diphosphoinositol-polyphosphate diphosphatase |
| EC 3.6.2 | In Sulfonyl-Containing Anhydrides |
| EC 3.6.2.1 | adenylylsulfatase |
| EC 3.6.2.2 | phosphoadenylylsulfatase |
| EC 3.6.3 | Acting on acid anhydrides; catalysing transmembrane |
| | movement of substances |
| EC 3.6.3.1 | Mg2+-ATPase |
| EC 3.6.3.2 | Mg2+-importing ATPase |
| EC 3.6.3.3 | Cd2+-exporting ATPase |
| EC 3.6.3.4 | Cu2+-exporting ATPase |
| EC 3.6.3.5 | Zn2+-exporting ATPase |
| EC 3.6.3.6 | H+-exporting ATPase |
| EC 3.6.3.7 | Na+-exporting ATPase |
| EC 3.6.3.8 | Ca2+-transporting ATPase |
| EC 3.6.3.9 | Na+/K+-exchanging ATPase |
| EC 3.6.3.10 | H+/K+-exchanging ATPase |
| EC 3.6.3.11 | Cl−-transporting ATPase |
| EC 3.6.3.12 | K+-transporting ATPase |
| EC 3.6.3.14 | H+-transporting two-sector ATPase |
| EC 3.6.3.15 | Na+-transporting two-sector ATPase |
| EC 3.6.3.16 | arsenite-transporting ATPase |
| EC 3.6.3.17 | monosaccharide-transporting ATPase |
| EC 3.6.3.18 | oligosaccharide-transporting ATPase |
| EC 3.6.3.19 | maltose-transporting ATPase |
| EC 3.6.3.20 | glycerol-3-phosphate-transporting ATPase |
| EC 3.6.3.21 | polar-amino-acid-transporting ATPase |
| EC 3.6.3.22 | nonpolar-amino-acid-transporting ATPase |
| EC 3.6.3.23 | oligopeptide-transporting ATPase |
| EC 3.6.3.24 | nickel-transporting ATPase |
| EC 3.6.3.25 | sulfate-transporting ATPase |
| EC 3.6.3.26 | nitrate-transporting ATPase |
| EC 3.6.3.27 | phosphate-transporting ATPase |
| EC 3.6.3.28 | phosphonate-transporting ATPase |
| EC 3.6.3.29 | molybdate-transporting ATPase |
| EC 3.6.3.30 | Fe3+-transporting ATPase |
| EC 3.6.3.31 | polyamine-transporting ATPase |
| EC 3.6.3.32 | quaternary-amine-transporting ATPase |
| EC 3.6.3.33 | vitamin B12-transporting ATPase |
| EC 3.6.3.34 | iron-chelate-transporting ATPase |
| EC 3.6.3.35 | manganese-transporting ATPase |
| EC 3.6.3.36 | taurine-transporting ATPase |
| EC 3.6.3.37 | guanine-transporting ATPase |
| EC 3.6.3.38 | capsular-polysaccharide-transporting ATPase |
| EC 3.6.3.39 | lipopolysaccharide-transporting ATPase |
| EC 3.6.3.40 | teichoic-acid-transporting ATPase |
| EC 3.6.3.41 | heme-transporting ATPase |
| EC 3.6.3.42 | b-glucan-transporting ATPase |
| EC 3.6.3.43 | peptide-transporting ATPase |
| EC 3.6.3.44 | xenobiotic-transporting ATPase |
| EC 3.6.3.45 | steroid-transporting ATPase |
| EC 3.6.3.46 | cadmium-transporting ATPase |
| EC 3.6.3.47 | fatty-acyl-CoA-transporting ATPase |
| EC 3.6.3.48 | a-factor-transporting ATPase |
| EC 3.6.3.49 | channel-conductance-controlling ATPase |
| EC 3.6.3.50 | protein-secreting ATPase |
| EC 3.6.3.51 | mitochondrial protein-transporting ATPase |
| EC 3.6.3.52 | chloroplast protein-transporting ATPase |
| EC 3.6.3.53 | Ag+-exporting ATPase |
| EC 3.6.4 | Acting on acid anhydrides; involved in cellular and |
| | subcellular movement |
| EC 3.6.4.1 | myosin ATPase |
| EC 3.6.4.2 | dynein ATPase |
| EC 3.6.4.3 | microtubule-severing ATPase |
| EC 3.6.4.4 | plus-end-directed kinesin ATPase |
| EC 3.6.4.5 | minus-end-directed kinesin ATPase |
| EC 3.6.4.6 | vesicle-fusing ATPase |
| EC 3.6.4.7 | peroxisome-assembly ATPase |
| EC 3.6.4.8 | proteasome ATPase |
| EC 3.6.4.9 | chaperonin ATPase |
| EC 3.6.4.10 | non-chaperonin molecular chaperone ATPase |
| EC 3.6.4.11 | nucleoplasmin ATPase |
| EC 3.6.5 | Acting on GTP; involved in cellular and subcellular |
| | movement |
| EC 3.6.5.1 | heterotrimeric G-protein GTPase |
| EC 3.6.5.2 | small monomeric GTPase |
| EC 3.6.5.3 | protein-synthesizing GTPase |
| EC 3.6.5.4 | signal-recognition-particle GTPase |
| EC 3.6.5.5 | dynamin GTPase |
| EC 3.6.5.6 | tubulin GTPase |
| EC 3.7 | Acting on Carbon-Carbon Bonds |
| EC 3.7.1 | In Ketonic Substances |
| EC 3.7.1.1 | oxaloacetase |
| EC 3.7.1.2 | fumarylacetoacetase |
| EC 3.7.1.3 | kynureninase |
| EC 3.7.1.4 | phloretin hydrolase |
| EC 3.7.1.5 | acylpyruvate hydrolase |
| EC 3.7.1.6 | acetylpyruvate hydrolase |
| EC 3.7.1.7 | b-diketone hydrolase |
| EC 3.7.1.8 | 2,6-dioxo-6-phenylhexa-3-enoate hydrolase |
| EC 3.7.1.9 | 2-hydroxymuconate-semialdehyde hydrolase |
| EC 3.7.1.10 | cyclohexane-1,3-dione hydrolase |
| EC 3.8 | Acting on Halide Bonds |
| EC 3.8.1 | In C-Halide Compounds |
| EC 3.8.1.1 | alkylhalidase |
| EC 3.8.1.2 | (S)-2-haloacid dehalogenase |
| EC 3.8.1.3 | haloacetate dehalogenase |
| EC 3.8.1.5 | haloalkane dehalogenase |
| EC 3.8.1.6 | 4-chlorobenzoate dehalogenase |
| EC 3.8.1.7 | 4-chlorobenzoyl-CoA dehalogenase |
| EC 3.8.1.8 | atrazine chlorohydrolase |
| EC 3.8.1.9 | (R)-2-haloacid dehalogenase |
| EC 3.8.1.10 | 2-haloacid dehalogenase (configuration-inverting) |
| EC 3.8.1.11 | 2-haloacid dehalogenase (configuration-retaining) |
| EC 3.9 | Acting on Phosphorus-Nitrogen Bonds |
| EC 3.9.1.1 | phosphoamidase |
| EC 3.10 | Acting on Sulfur-Nitrogen Bonds |
| EC 3.10.1.1 | N-sulfoglucosamine sulfohydrolase |
| EC 3.10.1.2 | cyclamate sulfohydrolase |
| EC 3.11 | Acting on Carbon-Phosphorus Bonds |
| EC 3.11.1.1 | phosphonoacetaldehyde hydrolase |
| EC 3.11.1.2 | phosphonoacetate hydrolase |
| EC 3.12 | Acting on Sulfur-Sulfur Bonds |
| EC 3.12.1.1 | trithionate hydrolase |
| EC 3.13 | Acting on Carbon-Sulfur Bonds |
| EC 3.13.1.1 | UDP-sulfoquinovose synthase |
| EC 3.13.1.2 | 5-deoxyribos-5-ylhomocysteinase |

In some embodiments, the changes to the natural ribosome to produce the modified ribosomes of the present invention are derived from the observation that the reverse reaction happens in the serine protease chymotrypsin, where histidine⁵⁷ and serine¹⁹⁵ exchange a proton through the substrate target protein, cleaving it.

A non-limiting example of the method for engineering a modified ribosomal active site of the present invention is to examine enzymes such as the reverse enzyme Kor cellulase or CesA glucosyltransferase to build a ribosomal active site capable of synthesizing cellulose or other polysaccharide.

### In Vitro Methods

Running DEP production in vitro requires one to duplicate most of the mechanisms of life in an external environment. Trying to run most of the mechanisms of life in some external “wash” environment can be difficult, but it eliminates the measures which can be required to separate a modified ribosomal production system from the natural synthesis processes of a living cell in a dual mode in vivo system.

In some embodiments, ribosomes can be harvested from cells and attached to endoplasmic reticulum equivalents. Suitable modified tRNAs and mRNA's are then created and mixed with the ribosomes. In, some embodiments, this process can be energetically driven using a supply of ATP. Substrate monomers are then mixed with the modified tRNAs. The mixture is then allowed to react and, in some embodiments, the DEPs produced can be later isolated from the reaction mixture. In some embodiments, a pedestal mount in vitro method can be used.

### In Vivo Methods

If in vivo methods are used the choice arises to attempt to have modified ribosomes coexist with natural ones within a cell or to supplant them entirely. The present invention is not directed to supplanting all function with the engineered ribosomes because the present invention strives to leave all the protein generating mechanisms alone while simultaneously synthesizing new bonds in a parallel system.

The present invention establishes a parallel system for polymer generation whose machinery does not interfere with or harm, as much as is possible, the normal base protein synthesis machinery of a cell and the rest of the normal base cellular metabolism. Viability of such reengineered cells during polymer synthesis is a preferred but long term viability of the cell is not required. For example, methods such as pulse production where the alternate systems start running, produce DEPs, and then kill the cell, can be used to produce DEPs.

This process of parallel metabolism with minimal modification is referred to as Dual Mode In vivo (DMIV). Once DMIV is established in a cell line, experimentation with the mechanism can be performed with a reduced chance of causing immediate cell death by breaking the normal base metabolism.

Further, explicit programming changes to structures using random variation similar to evolutionary processes, can be used to generate new DMIV products. If the parallel dual mode in vivo synthesis mechanism is non-fatal, then mutation can be used to explore the space of similar synthetic structures. The process of using mutations to explore an engineered space of possibilities while not transgressing on the normal base metabolism coded by the normal base genome is called Induced Parallel Mode Mutation (IPMM).

### Directed Element Polymers

The present invention is also directed to biomimetic structures, also known as DEPs. In some embodiments, the present invention is directed to producing DEPs on a nanotech scale.

The term nanotechnological refers to any fabrication technology in which objects are designed and built by the specification and placement of individual atoms or molecules or where at least one dimension is on a scale of nanometers, for example, producing a DEP with a single chemical bond. A nanotechnological structure refers to any structure produced using a nanotechnological process or to any structure that has at least one dimension on a scale of nanometers.

DEPs can be made using a living system comprising a modified ribosome to produce this new class of materials called directed element polymers. These new materials resemble proteins, in that they are structures that have a directed pattern imposed on them. Like proteins, each of these classes of polymers are made from a set of varying monomeric units, i.e. substrate molecules, that are specified by a nucleic acid template. In embodiments of the present invention, where the monomers used to form the polymer are not identical, the polymer will be a copolymer.

Biomimetic structures, also known as DEPs, encompassed by the present invention include any polymer not naturally utilized or produced in the mimicked biological processes. The directed elements may be linked together using chemical bonds and are, as such, not limited to the peptide bonds produced by the natural translational mechanism. For example, this invention is directed to the formation of the following bonds: I) bonds in natural polymers found in existing life forms, 2) bonds between monomers that are naturally occurring but are not traditionally found in life forms, 3) bonds between monomers that form polymers that are industrially produced, 4) bonds between high-energy monomers, or 5) any other chemical bond capable of linking monomers, either currently known or later discovered.

In some embodiments, the DEPs of the present invention can be any type of copolymer, including, but not limited to block copolymers, statistical copolymers, grafting copolymers, or alternating copolymers.

### Bond Selection

Each class of biomimetic structure, i.e., each DEP, is characterized by its polymerization bond. Each of these classes is based on some set of similar monomers that can be bonded together via that bond. Using thousands of bonds and producing millions of different polymeric chains, novel and cost-effective materials and substances can be produced using living systems comprising modified ribosomes that cannot, or currently are not, made by natural life forms or industrial methods. Their physical properties are dependent on the bond used and choice of monomers. In some embodiments, the biomimetic structure produced can be stiff or flexible; long or short: coiled or not coiled; globular, or combinations thereof.

Natural proteins have a multitude of uses because of their geometric properties. For example, because the peptide bond is flexible, the long polymer of amino acids acts like a piece of cooked spaghetti rather than a dry stiff piece. However, various other bonds will have different stiffnesses and resilience, leading to differences in flexibility and durability.

In some embodiments, the present invention is directed to DEPs having stiff bonds. In other embodiments, the present invention is directed to DEPs having flexible bonds.

Bond stiffness in the DEP can be used to determine the proper application for the DEP. For example, stiffer molecules may be better for use as structural or electronic materials, while more flexible ones could be designed to fold in some manner, exposing side groups in ways that enhance catalytic behavior.

In some embodiments, the folding characteristics of the DEP can be used to classify DEPs. As stated earlier, the definition of a protein is a sequenced polymerized string of amino acids. Theoretically being able to predict the folding and self bonding of polymerized amino acids is part of Proteomics. For each set of DEPs produced by the present invention there would be a specific scientific discipline dealing with how that class of molecules bends, folds and rebonds to itself.

### Applications for DEPs

Some uses of DEPs produced using the BIOP method are listed below in Table 2. These uses are exemplary and are not intended as limiting as one of skill in the art will recognize a wide variety of other uses for the DEPs produced using this invention. The term “computational” as used in Table 2 means the use of enzyme systems to process chemical signals to have some effect based on a logical computation of these signals, for example, the G protein cascade in cells.

TABLE 2
Applications for DEPs

| Type | Purpose |
|---|---|
| Catalytic | facilitate chemical reactions |
| Computational | input reactions change of its other properties |
| Transportational | attaches to other substances |
| Electrical Insulators | retard electron transport |
| Electrical Conductors | allow electron transport |
| Effector | creates movement |
| Transducer | emit or absorb energy |
| Structural | forms mechanical structures |

### Isolation of DEPs

DMIV is an example of a method of isolating artificial subsystems from interacting with natural subsystems. DMIV, along with other methods of locality, can be used to keep the modifications (e.g. DEPs) from upsetting the balance between natural and unnatural polymers. One method to isolate the artificial subsystems is through analogy to the use of computers.

In computer science, many techniques of isolation are used to simplify both the actual coding of the solution as well as abstract meta-level structures describing the solution of the problem. Computer science, as opposed to the physical sciences, is an artificial intellectual construct but has actual code that runs on real computers. In the physical sciences, the machinery or operating code is visualized, but the meta-level description is not comprehended. Code is encapsulated into Subroutines. Variables can be Global if necessary, but are more useful if kept Local to the subroutines. Thus was born the lambda calculus and its association with the class of recursive functions. To further restrict the interaction of code with itself, Operating Systems, Processes. Virtual Machines, Address Spaces. Control Stacks, and Local Environments were invented.

Biological organisms invented membranes to literally encapsulate the reactions it was trying to control and self-perpetuate. While cells appear to be small droplets of ocean of three billion years ago, they are also small tanks limiting the molecules that are interacting to execute the life machine into a small volume across which iterations would probabilistically occur. As evolution has progressed, the pitfalls of universal interaction caused further evolution of localizing structures. Internal membranes such as endoplasmic reticulum and Golgi apparatus led to organelles such as the nucleus. Symbiotic evolution, as described by Margulis, led to the construction of organelles such as the mitochondrion and the chloroplast.

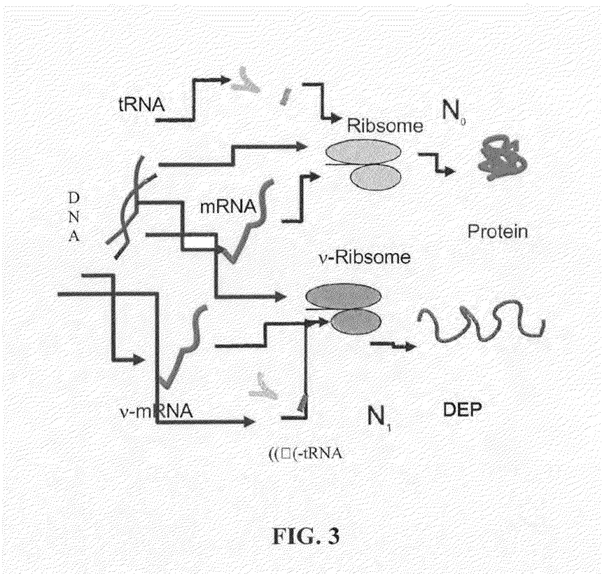

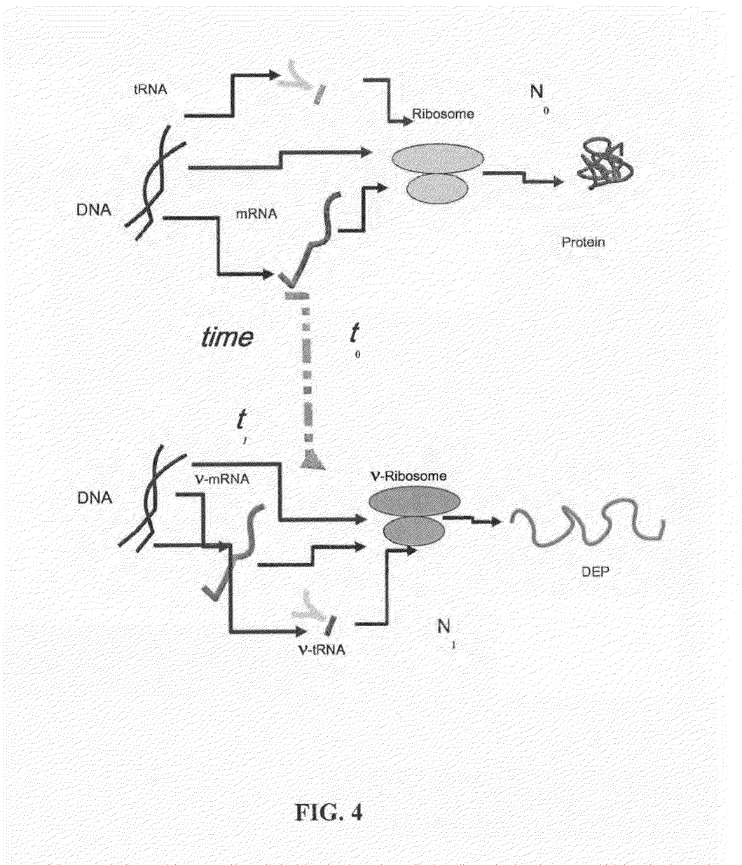

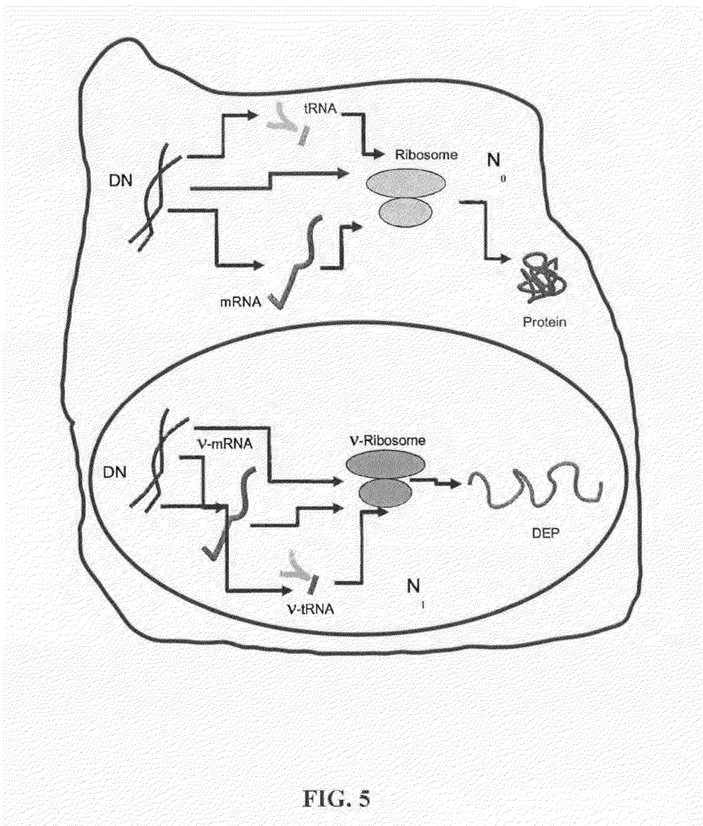

Therefore, there are several ways these natural/unnatural processes can be constructed. For Dual Mode In Vivo (DMIV) (shown in FIG. 3) processes, the processes are set up in parallel to the natural cellular processes, allowing for simultaneous operation. In Temporal Mode In Vivo (TMIV) (shown in FIG. 4), the alternate processes are set up in parallel to the natural processes; however, the processes are functioning at a different time. Lastly, Isolated Mode In Vivo (IMIV) (shown in FIG. 5) allows for the alternate processes to be set up in parallel to the natural processes, however, they occur in membrane isolated spaces. The interactions can be logically isolated with tagged and coded unique interacting regions. To accomplish this, the existing organelle structures are modified that have created partially isolated regions of the cell. While these organelles originally formed by symbiosis, their purpose is keep certain reactions isolated from the cytosol.

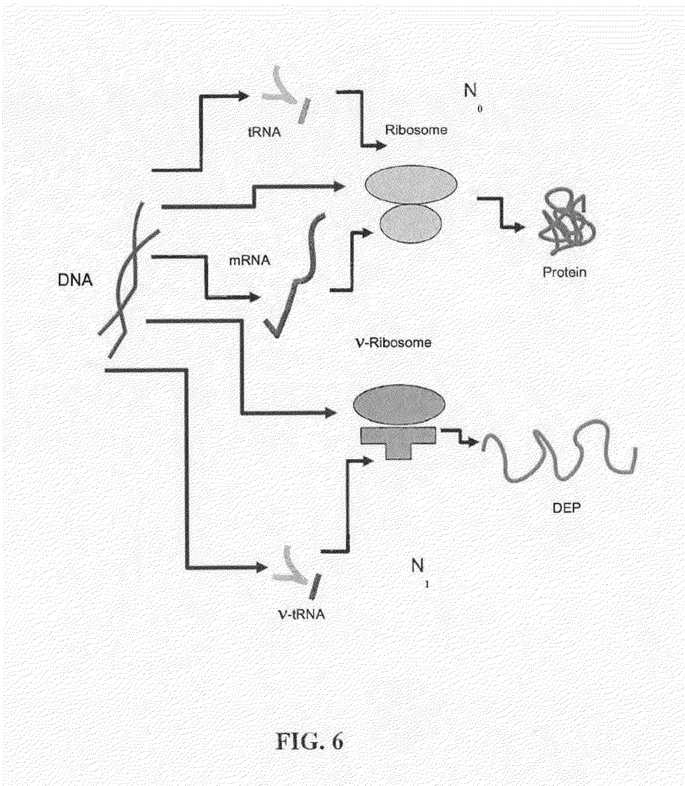

DMIV uses a modified small ribosomal component to read specified mRNA strings that have an alternative leader sequence, in order to attach to the small ribosomal component. In Autologous Mode In Vivo (AMIV) (shown in FIG. 6), the in vivo process are set up in parallel to natural process that do not use mRNA for input, but specifies tRNA's by finite state machine states of its internal structure.

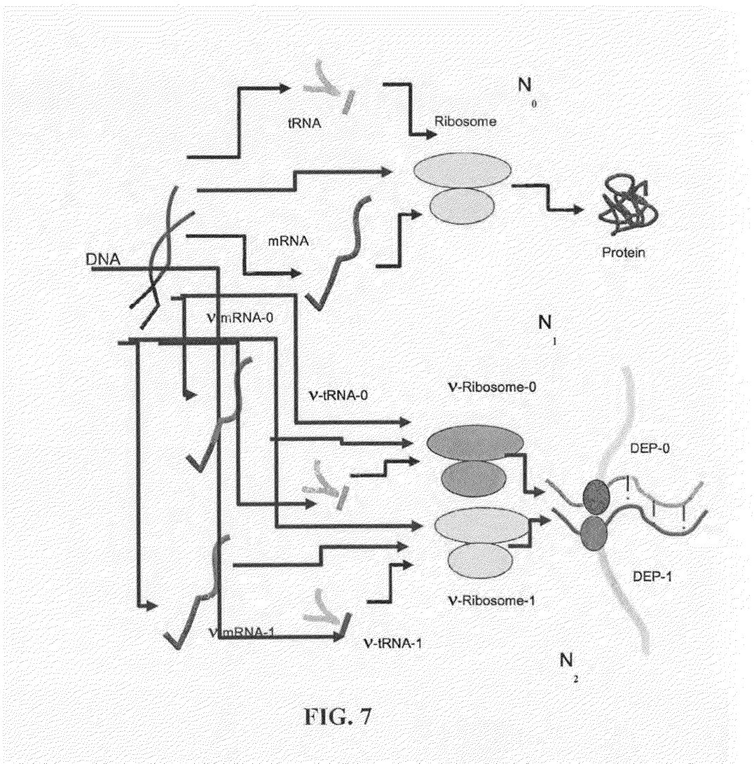

DMIV can also be generalized to allow more than one artificial pathway. In Multiple Mode In Vivo (MMIV) (shown in FIG. 7), multiple in vivo processes are set up in parallel to natural processes, allowing simultaneous operation. By having multiple simultaneous production systems, structures are made as combinations of different polymers. Modified translocons are reengineered to cluster and attract multiple ribosomes to produce simultaneous polymer chains chains that interact into larger structures.

In one embodiment of the invention, the isolation of reengineered biomimetic structures from natural ribosomal mechanisms is performed by DMIV. In DMIV, the DNA source code for both reactive elements and target polymers apply to both the natural and alternative pathways, but all further steps of the protein synthesis mechanism are duplicated and the copy modified. Each step in the synthesis process has a new analog: new mRNAs with alternative initiator leaders: new tRNAs to bind the alternative monomers; new synthases to catalyze the monomer/tRNA binding; new content sensitive reader on the small ribosomal component to only attach to mRNA; new synthesizing site on the large ribosomal component as well as ribosomal mating points and tunnel work; new DEP segregation methods for storing, secreting or directly secreting DEPs by modifying signal recognition protein (SRP) carriers, chaperon proteins, the SR61 translocon and the various prokaryote membrane translocons such as secDF and their chloroplast analogs TOC and TIC and the mitochondiral analog TIM.

For Dual Mode In Vivo (DMIV), the processes are set up in parallel to the natural cellular processes, allowing for simultaneous operation. In Temporal Mode In Vivo (TMIV), the alternate processes are set up in parallel to the natural processes, however the processes are functioning at different time. Lastly, isolated Mode In Vivo (IMIV) allows for the alternate processes to be set up in parallel to the natural processes, however, they occur in membrane isolated spaces.

In certain embodiments of the invention, a lipid layer of any size or depth can be created. Some examples of lipid layers include, but are not limited to a lipid monolayer (e.g., POPC liposomes), lipid bilayer (e.g. hydrogel containing lipophilic groups (like alkanes) to anchor liposomes: Lahiri's amphiphilic anchor lipid surface which binds lipid bilayers (Langmuir 16:7805-7810 (2000)): proteolipid bilayer (attach detergent solubilized receptors to surface in an oriented manner and reconstitute lipid membrane around immobilized receptors using lipid micelles while removing detergent (Analyt. Biochem. 300:132-8 (2000)); use membrane fractions containing over expressed receptors). Lipid surfaces can also be as described in, e.g., Baird et al. Analyt. Biochem. 310:93-99(2002): Stenlund et al. Analyt. Biochem. 316:243-250 (2003); Abdiche & Myszka, Analyt. Biochem. 328:233-243 (2004); Ferguson et al., Bioconjugate Chem. 16:1457-1483 (2005); U.S. Pat. No. 6,756,078.

New organelle structures can be formed to isolate the interactions, either by alternate structures such as the nucleolus or by using the precepts noted by Margulis and create life within life to isolate the particular structures operating, while using the environment of the surrounding cell to support the operation of the partially isolated mechanism. However, to use these new alternature life structures, limits must be set on the number of cell divisions to limit runaway growth, other multiple growth limiting mechanisms must be used. The most straightforward approach is to require that reengineered organisms need to be supplied with essential growth factors necessary for their survival.

### Alternative Input Methods

DMIV uses a modified small ribosomal component to read specified mRNA strings that have an alternative leader sequence, in order to attach to the small ribosomal component. For applications where the DEP is a simple polymer or one with a simple repeating pattern, the reengineered small ribosomal component functioning as an mRNA reader is replaced by a reengineered ribosomal component that has the polymer pattern encoded in its structure. One example of this method is Autologous Mode In Vivo (AMIV), wherein the in vivo synthetic process is set up in parallel to a natural process that does not use mRNA for input but specifies tRNAs by finite state machine states of its internal structure.

DMIV can be generalized to allow for more than one artificial pathway. In Multiple Mode In Vivo (MMIV), multiple in vivo processes are set up in parallel to natural processes, allowing simultaneous operation. By having multiple simultaneous production systems, one is able to make structures that are combinations of different polymers. Modified translocons are reengineered to cluster and attract multiple ribosomes to produce simultaneous polymer chains chains that interact into larger structures.

### Macro Ribosomal Assembly Structures

In one embodiment of the invention, macro ribosomal assembly structures are used for the production of general purpose nanotechnological devices. Particular unfolded and folded DEPs can be used as parts for nanotechnological devices, but proper assembly is necessary for functioning. While new assembly organelles of the complexity of ribosomes and spliceosomes will need to be invented in the future, a first step in that direction is the use of ribosome assemblies using ribosomes operating in conjunction to create structures one level up from single string DEPs.

To achieve the macro synthesis of DEPs, more than one artificial pathway can be constructed in one cell. This generalization of DMIV to produce more than one additional bond based polymer is called Multiple Mode In Vivo production. In MMIV multiple in vivo processes set up in parallel to natural processes, allowing simultaneous operation. By having multiple simultaneous production systems, structures are made that are combinations of different polymers.

In order to generate these combinations, assemblies of ribosomes can be arrayed together to produce simultaneous chains. These arrays can either be in a static relationship with each other or connected in a physically dynamic array. Simple arrays can produce parallel polymer chains that are designed to bond together at specific points. The folding patterns of the individual polymers are changed by the interaction of two or more chains. Dynamic ribosome arrays can be used to produce multiple polymer chain macromolecules that have a physical tension imparted by the energy investment of the dynamic array. Such multiple chain molecules can be called nano-ropes and nano-braids.

Engineered cells can either be run in DMIV or MMIV. In either case, engineered ribosomes can either be free floating, attached to membranes or clustered in parallel groups to generate interacting DEPs. When clustered, the cluster assemblies can also either be free floating or attached to membranes. In one embodiment of the invention, the modified translocons are reengineered to cluster into larger structures in a membrane. These structures can attract multiple ribosomes to produce simultaneous polymer chains that interact. In another embodiment, free floating ribosomes engage in ribosome-ribosome attachment to produce simultaneous polymer chains chains that interact. Ribosomes are reengineered to have attachment point proteins incorporated along the equator orthogonal to the polymer tunnel. Ribosomes are then either attached via these points or using larger interstitial materials. For static arrangements of multiple ribosomes, the attachments are relatively simple. For dynamic arrangements use of actin, myosin and components of microtubules, centromeres and flagellar motors are used to ratchet around each other.

### Energy Related Methods

The polymers produced by the embodiments of the invention can compete with the petrochemical industry for types of substrates used in the synthesis process. The energy to drive the polymerization process is obtained from biological sources. This can either be through oxidative processes of normal cell metabolism or it can use the photosynthetic abilities of algae and plants to capture solar energy for metabolic use. Once organisms acquire the energy necessary to drive DEP synthesis, excess energy can be stored as produced polymers similar to the investment of energy into production of the polysaccaride cellulose. In fact, many fuels used currently are, in fact, short polymers.

Having generally described this invention, a further understanding can be obtained by reference to the examples provided herein. These examples are for purposes of illustration only and are not intended to be limiting.

### EXAMPLES

The present invention can be used to produce either naturally occurring polymers, biomimetic structures, or nanotechnological structures. To use the present invention to produce natural polymers, such as cellulose, the existing natural ribosome system of protein production is examined to identify points which need to be modified to produce the desired polymer. Further, other naturally occurring polymers can be produced using the present invention after an examination of the process for making and degrading the biological polymers such as sugars, starches and other polysaccharides, polylactic acid, polyglycolic acid, fatty acids, polytriglycerides, or polyhydroxyalkanoates.

More complex and alien DEPs are those that use variant monomers with either high energy carbon bonds such acetylene or pure carbon bonds such as graphite or diamond, or use of esoteric elements similar to carbon such as silicon or germanium. The chemical properties of such DEPs are difficult to predict but one of skill in the art could determine them using chemical modeling programs.

In some embodiments, the method of alternate polymer production requires certain changes to be implemented in a host cell, such as E. coli. Importantly, this method leaves the DNA operational mechanisms largely untouched except for the addition of the new programmed coding material required to produce any or all of the following: the nucleic acid template, the modified tRNAs, the modified tRNA synthetases, or the modified ribosomes. Similarly this method leaves the operation of the natural mRNA intact, while creating a novel class of mRNA to feed information to the modified ribosomes. In some embodiments, this separation between natural mRNAs and the ones used in the present invention is accomplished by using a leader recognition induction area that is defined on the ribosome.

After establishing the proper DNA and mRNA templates, a new class of tRNA used to bring the substrate molecules to the modified ribosomes will be created. The attachment of the substrate molecule to the modified tRNA will require, in some embodiments, the creation of a novel tRNA synthetase. The novel tRNA and a tRNA synthetase interact to bring the substrate molecule to the modified ribosome.

The present invention requires modification of a natural ribosome to create the modified ribosomes of the present invention. Such modifications include changing the key catalytic site of the ribosome, for example the A2451 base, to accommodate the formation of a new bond. The body of the ribosome and exit tunnel can be modified structurally to ensure that the generated polymer does not clog the ribosomal exit tunnel. These changes will be implemented by modifying the DNA sequences used to create natural ribosomes and then allowing this DNA to be expressed in a selected host cell.

Within the host cell, the attachment docking areas that attach the ribosome to membranes, such as the endoplasmic reticulum, will have to be modified. Further, the attachment sites on some of the membranes will have to be changed to have cellular mechanisms to process, segregate, excrete, or tolerate the produced polymer. In some embodiments, the DEP will be stored in special vacuoles to prevent injury to the host cell.

### Example 1

### Peptide Bond

### Protein

The example method described previously can be used to generate novel proteins using the peptide bond in host cells which do not ordinarily synthesize these proteins. Some of the key components for creating a dual mode in vivo system for creating these novel proteins are summarized in Table 3.

TABLE 3
Base Example of Polypeptide

| Property | Value |
|---|---|
| Bond | Peptide |
| Monomer Set | About 20 (all amino acids) |
| Codon Length | 3 |
| Substrate Viability Compatibility | Total |
| Active Site Known | Yes |
| Similar Synthetic Enzymes Known | No |
| Degradative Enzymes Known | Yes (e.g., chymotrypsin) |
| Restrictive Leader Class | Type 0 (base, i.e. none) |
| tRNA Possible for Substrates | Yes |
| Tunnel Rework Required | No |
| Product Viability Compatibility | Yes (Exceptions: various natural |
| | toxins) |

### Example 2

### Cellulose

The following describes a process which can be used to design a method of producing cellulose using the present invention. The designations of first, second, etc. are provided for organizational purposes only. As one of skill in the art will recognize, most steps can be performed in any order to design the production method described below.

First, researchers identify the bond needed to synthesize the DEP desired, for example the glycoside bond used in cellulose. Second, changes needed in the structure and function of the ribosomal active site are identified to produce a similar but reverse direction catalytic mechanism based on examining the reaction of a hydrolyzing enzyme such as Kor cellulase.

Third, for this example, the tRNAs used will incorporate a codon system of 3 nucleotides because this particular DEP does not require linking a vast number of different substrates but instead is composed of simple sugars only, e.g., β-D-Glucose. While a cellulose polymer consisting of only β-D-Glucose is provided as an example, one of skill in the art will recognize that various isomers of cellulose or cellulose-like compounds can be produced by the method of the present invention.

Fourth, tRNA synthetases are designed to attach the simple sugars used as substrate molecules to the modified tRNAs used in the present invention. In some embodiments, following the naming convention of the aminoacyl tRNA synthetases, the synthetases of this embodiment of the present invention can be termed glucoglycosidic synthetases because they will be adding a glucose molecule (instead of an amino acid) to the modified tRNA via a glycosidic bond (instead of an acyl bond). However, any bond can be used to attach the glucose molecules to the modified tRNA not only a glycosidic bond. Fifth, modified tRNAs are designed to carry the simple sugars to the modified ribosome.

Sixth, the reading function of the 30s ribosomal subunit is designed so that the modified ribosome can function parallel to the natural system. As part of designing this parallel synthetic system. mRNA with a unique leader will be designed.

Seventh, the structure of the natural ribosomal tunnel is examined and redesigned so that the DEP, e.g., cellulose, can pass through it without clogging the tunnel.

Eighth, a system for isolating the product, e.g., cellulose, is designed. Such a system can involve isolating the product from the rest of the host cell, particularly useful if the DEP produced is toxic or otherwise harmful to the cell, and then later removing the product or a method of transporting the DEP through the cellular membrane after it has been produced.

Finally, the design changes identified in the preceding steps are translated into a nucleic acid template and inserted into a host cell to begin production of the DEPs.

The example production method of the present invention described previously can be used to generate cellulose in host cells which do not ordinarily synthesize cellulose. Some of the key components for creating a dual mode in vivo system for creating cellulose are summarized in Table 4.

TABLE 4
Example of Cellulose

| Property | Value |
|---|---|
| Bond | Glycosidic |
| Monomer Set | 1 (β-D-glucose) |
| Codon Length | 1 to 20 |
| Substrate Viability Compatibility | Yes |
| Active Site Known | No |
| Similar Synthetic Enzymes Known | Yes (CesA glucosyltransferase) |
| Degradative Enzymes Known | Yes (Kor cellulase) |
| Restrictive Leader Class | Type I |
| tRNA Possible for Substrates | Yes |
| Tunnel Rework Required | Yes |
| Product Viability Compatibility | Not available |

### Example 3

### Polylactic Acid

### PLA

The example method described previously can be used to generate PLA in host cells which do not ordinarily synthesize PLA. Some of the key components for creating a dual mode in vivo system for creating PLA are summarized in Table 5.

TABLE 5
Example of PLA

| Property | Value |
|---|---|
| Bond | Any |
| Monomer Set | 1 |
| Codon Length | 1 to 20 |
| Substrate Viability Compatibility | Yes |
| Active Site Known | No |
| Similar Synthetic Enzymes Known | Yes |
| Degradative Enzymes Known | Yes |
| Restrictive Leader Class | Type I |
| tRNA Possible for Substrates | Yes |
| Tunnel Rework Required | Yes |
| Product Viability Compatibility | Not available |

### Example 4

### Polyethylene

### PE

The example method described previously can be used to generate PE in host cells which do not ordinarily synthesize PE. Some of the key components for creating a dual mode in vivo system for creating PE are summarized in Table 6.

TABLE 6
Example of PE

| Property | Value |
|---|---|
| Bond | Any |
| Monomer Set | 1 |
| Codon Length | 1 to 20 |
| Substrate Viability Compatibility | Not available |
| Active Site Known | No |
| Similar Synthetic Enzymes Known | No |
| Degradative Enzymes Known | Yes |
| Restrictive Leader Class | Type I |
| tRNA Possible for Substrates | Yes |
| Tunnel Rework Required | Yes |
| Product Viability Compatibility | Not available |

### Example 5

### Polysilane

The example method described previously can be used to generate polysilane in host cells which do not ordinarily synthesize polysilane. Some of the key components for creating a dual mode in vivo system for creating polysilane are summarized in Table 7.

TABLE 7
Example of PS

| Property | Value |
|---|---|
| Bond | Any |
| Monomer Set | About 5 |
| Codon Length | 1 to 20 |
| Substrate Viability Compatibility | Not available |
| Active Site Known | No |
| Similar Synthetic Enzymes Known | No |
| Degradative Enzymes Known | Yes |
| Restrictive Leader Class | Type I |
| tRNA Possible for Substrates | Yes |
| Tunnel Rework Required | Yes |
| Product Viability Compatibility | Not available |

### Example 6

### Organometallic Polymers

The example method described previously can be used to generate organometallic polymers in host cells which do not ordinarily synthesize organometallic. An organometallic compound is an organic compound comprising a metal. In some embodiments, the metal is directly bound to a carbon atom. Some of the key components for creating a dual mode in vivo system for creating an organometallic polymer are summarized in Table 8.

TABLE 8
Example of Organometallic Compounds

| Property | Value |
|---|---|
| Bond | Any |
| Monomer Set | About 10 |
| Codon Length | 1 to 20 |
| Substrate Viability Compatibility | Not available |
| Active Site Known | No |
| Similar Synthetic Enzymes Known | No |
| Degradative Enzymes Known | No |
| Restrictive Leader Class | Type I |
| tRNA Possible for Substrates | Yes |
| Tunnel Rework Required | Yes |
| Product Viability Compatibility | Not available |

Having now fully described the present invention in some detail by way of illustration for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents, and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of producing a nanotechnological or biomimetic structure comprising:
(a) forming a mixture comprising:
(i) a modified ribosome,
(ii) natural or unnatural coding material,
(iii) natural or unnatural substrate molecules for assembly into a nanotechnological or biomimetic structure, and
(iv) natural or unnatural factors required for synthesis of the nanotechnological or biomimetic structure during the initiation, elongation, or termination phases of assembly, and
(b) reacting the mixture under conditions capable of producing a nanotechnological or biomimetic structure,
wherein a nanotechnological or biomimetic structure is produced.

2. The method of claim 1, further comprising the step of:
(c) isolating the nanotechnological or biomimetic structure.

3. The method of claim 1, wherein the structure produced has at least one dimension on a scale of nanometers.

4. The method of claim 1, wherein the conditions comprise nonphysiological conditions selected from the group consisting of elevated or reduced pressure, elevated or reduced temperature, elevated or reduced pH, and combinations thereof.

5. The method of claim 1, wherein the unnatural coding material of the mixture comprises modified nucleoside bases or non-nucleoside base replacements.

6. The method of claim 1, wherein the substrate of the mixture comprises natural or unnatural amino acids, modified natural or unnatural amino acids, non-amino acid molecules suitable for assembly into a nanotechnological or biomimetic structure, or combinations thereof.

7. The method of claim 6, wherein the substrate of the mixture further comprises a metal.

8. The method of claim 1, wherein the natural or unnatural factors of the mixture further comprise an acceptor molecule selected from the group consisting of natural tRNA, unnatural tRNA, an acceptor molecule capable of interacting with the coding material of the mixture to assemble substrate molecules into a nanotechnological or biomimetic structure, and combinations thereof.

9. The method of claim 8, wherein the acceptor molecule of the mixture interacts with coding material of the mixture in sequences greater or less than three bases or base replacements in length.

10. The method of claim 1, wherein the modified ribosome of the mixture is capable of acting as an acceptor molecule for assembling the substrates of the mixture into a nanotechnological or biomimetic structure.

11. The method of claim 1, wherein the natural or unnatural factors of the mixture comprise natural or unnatural catalysts that transfer substrates to acceptor molecules.

12. The method of claim 1, wherein the reacting the mixture in step (b) comprises an in vivo, in vitro, or a cell-free system.

13. The method of claim 1, wherein the modified ribosome, natural or unnatural coding material, natural or unnatural factors of the mixture, and combinations thereof are introduced into the mixture using a genetic delivery system.

14. The method of claim 13, wherein the genetic delivery system is a virus, plasmid, or other coding material and wherein the conditions are appropriate for expression of the coding material.

15. A modified ribosome capable of assembling a nanotechnological or biomimetic structure.

16. The modified ribosome of claim 15, wherein the ribosome is modified from a natural ribosome.

17. The modified ribosome of claim 16, wherein the modified ribosome comprises ribosomal subunits in natural or unnatural combinations.

18. The modified ribosome of claim 17, wherein the ribosomal subunits comprise natural or unnatural mixtures.

19. The modified ribosome of claim 18, wherein the modified ribosome comprises an unnatural molecular size, molecular weight, or combination thereof.

20. A dual mode in vivo method of producing a nanotechnological or biomimetic structure in a host cell comprising:
(a) forming a mixture comprising:
(i) a modified ribosome(s),
(ii) a natural ribosome(s),
(iii) natural or unnatural coding material,
(iv) natural or unnatural substrate molecules for assembly into a nanotechnological or biomimetic structure, and
(v) natural or unnatural factors required for synthesis of the nanotechnological or biomimetic structure during the initiation, elongation, or termination phases of assembly, and
(b) reacting the mixture of step (a) under conditions capable of producing a nanotechnological or biomimetic structure,
wherein a nanotechnological or biomimetic structure is produced in a host cell.

21. The method of claim 20, further comprising the step of:
(c) isolating the nanotechnological or biomimetic structure.

22. The method of claim 20, further comprising the steps of:
(c) mutating the parallel dual mode in vivo pathway of the host cell to produce a different nanotechnological or biomimetic structure from the biomimetic or nanotechnological structure produced in a non-mutated host cell.

23. The method of claim 20, wherein the conditions comprise nonphysiological conditions selected from the group consisting of elevated or reduced pressure, elevated or reduced temperature, elevated or reduced pH, and combinations thereof.

24. The method of claim 20, wherein the host cell is E. coli.

25. The method of claim 20, wherein the unnatural coding material of the mixture comprises modified nucleoside bases or non-nucleoside base replacements.

26. The method of claim 20, wherein the substrate of the mixture comprises natural or unnatural amino acids, modified natural or unnatural amino acids, non-amino acid molecules suitable for assembly into a nanotechnological or biomimetic structure, or combinations thereof.

27. The method of claim 26, wherein the substrate of the mixture further comprises a metal.

28. The method of claim 20, wherein the natural or unnatural factors of the mixture further comprise an acceptor molecule selected from the group consisting of natural tRNA, unnatural tRNA, an acceptor molecule capable of interacting with the coding material of the mixture to assemble substrate molecules into a nanotechnological or biomimetic structure, and combinations thereof.

29. The method of claim 28, wherein the acceptor molecule of the mixture interacts with coding material of the mixture in sequences greater or less than three bases or base replacements in length.

30. The method of claim 20, wherein the modified ribosome of the mixture is capable of acting as an acceptor molecule for assembling the substrates of the mixture into a nanotechnological or biomimetic structure.

31. The method of claim 20, wherein the natural or unnatural factors of the mixture comprise natural or unnatural catalysts that transfer substrates to acceptor molecules.

32. The method of claim 20, wherein the modified ribosome, natural or unnatural coding material, natural or unnatural factors of the mixture, and combinations thereof are introduced into the mixture using a genetic delivery system.

33. The method of claim 32, wherein the genetic delivery system is a virus, plasmid, or other coding material and wherein conditions are appropriate for expression of the coding material.

34. A method of modifying a ribosome, the method comprising:
(a) observing the degradative process used by hydrolyzing enzymes to break a chemical bond; and
(b) modifying the active site of a natural ribosome to produce the reverse reaction of the observed degradative process.

35. The method of claim 34, wherein the reverse reaction is designed to synthesize cellulose.

36. The method of claim 34, wherein the reverse reaction is designed to synthesize polylactate.

37. An unnatural acceptor molecule comprising a molecule capable of transporting a substrate to a modified ribosome.

38. The unnatural acceptor molecule of claim 37, wherein the unnatural acceptor molecule is an unnatural tRNA.

39. The unnatural acceptor molecule of claim 37, wherein the codon sequence length exceeds three nucleotides but is less than ten nucleotides in length.

40. A nanotechnological or biomimetic structure produced using the method of claim 1.

41. The biomimetic structure of claim 40, wherein the structure is a polymer or macrocyclic molecule.

42. A nanotechnological or biomimetic structure produced using the method of claim 6.

43. The biomimetic structure of claim 42, wherein the structure is a polymer or macrocyclic molecule.

44. A nonhuman organism comprising a modified ribosome, wherein the nonhuman organism is capable of synthesizing a nanotechnological or biomimetic structure.

45. A host cell comprising a modified ribosome, wherein the host cell is capable of synthesizing a nanotechnological or biomimetic structure.

46. A method of producing a nanotechnological or biomimetic structure in a host cell comprising:
(a) forming a mixture comprising:
(i) a modified ribosome(s),
(ii) a natural ribosome(s),
(iii) natural or unnatural coding material,
(iv) natural or unnatural substrate molecules for assembly into a nanotechnological or biomimetic structure, and
(v) natural or unnatural factors required for synthesis of the nanotechnological or biomimetic structure during the initiation, elongation, or termination phases of assembly, and
(b) reacting the mixture of step (a) under conditions capable of producing a nanotechnological or biomimetic structure,
(c) isolating the nanotechnological or biomimetic structure by storing, secreting or directly secreting the nanotechnological or biomimetic structure,
wherein a nanotechnological or biomimetic structure is produced in a host cell.

47. The method of claim 46, wherein the isolation is performed by temporal or spatial isolation.

48. The method of claim 47, wherein said isolation method comprises modifying signal recognition protein (SRP) carriers, modifying chaperone proteins or modifying cellular translocons.

49. The method of claim 46, further comprising the steps of:
(d) mutating the parallel dual mode in vivo pathway of the host cell to produce a different nanotechnological or biomimetic structure from the biomimetic or nanotechnological structure produced in a non-mutated host cell.

50. The method of claim 46, wherein the conditions comprise nonphysiological conditions selected from the group consisting of elevated or reduced pressure, elevated or reduced temperature, elevated or reduced pH, and combinations thereof.

51. The method of claim 46, wherein the host cell is E. coli.

52. The method of claim 46, wherein the unnatural coding material of the mixture comprises modified nucleoside bases or non-nucleoside base replacements.

53. The method of claim 46, wherein the substrate of the mixture comprises natural or unnatural amino acids, modified natural or unnatural amino acids, non-amino acid molecules suitable for assembly into a nanotechnological or biomimetic structure, or combinations thereof.

54. The method of claim 53, wherein the substrate of the mixture further comprises a metal.

55. The method of claim 46, wherein the natural or unnatural factors of the mixture further comprise an acceptor molecule selected from the group consisting of natural tRNA, unnatural tRNA, an acceptor molecule capable of interacting with the coding material of the mixture to assemble substrate molecules into a nanotechnological or biomimetic structure, and combinations thereof.

56. The method of claim 55, wherein the acceptor molecule of the mixture interacts with coding material of the mixture in sequences greater or less than three bases or base replacements in length.

57. The method of claim 46, wherein the modified ribosome of the mixture is capable of acting as an acceptor molecule for assembling the substrates of the mixture into a nanotechnological or biomimetic structure.

58. The method of claim 57, wherein the modified ribosome is attached to a membrane or is free-floating within the cell.

59. The method of claim 46, wherein the natural or unnatural factors of the mixture comprise natural or unnatural catalysts that transfer substrates to acceptor molecules.

60. The method of claim 46, wherein the modified ribosome, natural or unnatural coding material, natural or unnatural factors of the mixture, and combinations thereof are introduced into the mixture using a genetic delivery system.

61. The method of claim 60, wherein the genetic delivery system is a virus, plasmid, or other coding material and wherein conditions are appropriate for expression of the coding material.

62. The method of claim 47, wherein the spatial isolation comprises separation by enclosure in a membraneous structure.

63. The method of claim 62, wherein the membraneous structure is a lipid bilayer.
